(19) United States
(12) Patent Application Publication
(10) Pub. No.: US 2021/0128689 A1
(43) Pub. Date: May 6, 2021

(54) COMPOSITIONS AND METHODS FOR TREATING ALLERGIC DISORDERS

(71) Applicants: Brian Kim, St. Louis, MO (US); Madison Mack, St. Louis, MO (US); Jonathan Brestoff Parker, St. Louis, MO (US)

(72) Inventors: Brian Kim, St. Louis, MO (US); Madison Mack, St. Louis, MO (US); Jonathan Brestoff Parker, St. Louis, MO (US)

(73) Assignee: Washington University, St. Louis, MO (US)

(21) Appl. No.: 17/072,358

(22) Filed: Oct. 16, 2020

Related U.S. Application Data
(60) Provisional application No. 62/915,793, filed on Oct. 16, 2019.

(51) Int. Cl. A61K 38/20; A61K 38/17; A61K 39/395; C07K 16/28; C07K 16/24; A61P 17/00; A61P 37/08

(52) U.S. Cl. CPC A61K 38/2086; A61K 38/1793; A61K 39/3955; C07K 16/2866; C07K 16/247; C07K 16/244; A61K 2039/505; A61P 17/00; A61P 37/08; C07K 2317/31; C07K 2319/30; C07K 2317/75; C07K 2317/76; A61K 38/2026

(57) ABSTRACT

Among the various aspects of the present disclosure is the provision of compositions and methods for the treatment of allergic disorders. In some embodiments, the allergic disorder is atopic dermatitis or related allergic disorder thereof. In some embodiments, the method comprises administration of a therapeutically effective amount of an NK cell-stimulating agent, such as an IL-15 superagonist.

### CROSS-REFERENCE TO RELATED APPLICATIONS

This application claims priority from U.S. Provisional Application Ser. No. 62/915,793 filed on 16 Oct. 2019, which is incorporated herein by reference in its entirety.

### STATEMENT REGARDING FEDERALLY SPONSORED RESEARCH OR DEVELOPMENT

This invention was made with government support under AR070116 awarded by the National Institutes of Health. The government has certain rights in the invention.

### MATERIAL INCORPORATED-BY-REFERENCE

Not applicable.

### FIELD OF THE INVENTION

The present disclosure generally relates to treatment of allergic disorders, such as atopic dermatitis (AD).

### SUMMARY OF THE INVENTION

Among the various aspects of the present disclosure is the provision of compositions and methods for treatment of allergic disorders.

An aspect of the present disclosure provides for a method of increasing an NK cell population or function in a subject having an allergic disorder, comprising administering an NK cell-stimulating agent to the subject.

In some embodiments, a therapeutically effective amount can be an amount effective to (i) increase an NK cell level or function in the subject compared to the NK cell level or function in a control not having the allergic disorder, (ii) increase the NK cell level or function in the subject compared to the NK cell level or function of the subject before being administered the NK cell-stimulating agent; or (iii) increase the NK cell level to a level greater than 97.5 percentile.

In some embodiments, increasing NK cell level or function in the subject treats or prevents symptoms associated with the allergic disorder.

In some embodiments, the allergic disorder is associated with NK cell level or function depletion.

In some embodiments, the allergic disorder is selected from atopic dermatitis (AD), eczema, food allergy, asthma, an eosinophilic esophagitis or eosinophilic gastrointestinal disorder, a deficiency in type 1 immunity, allergic rhinitis, chronic rhinosinusitis, or a related allergic disorder thereof.

In some embodiments, the allergic disorder is atopic dermatitis (AD).

In some embodiments, the subject has less than a 97.5 percentile level of NK cells before being administered the NK cell-stimulating agent.

In some embodiments, the NK cell-stimulating agent comprises an IL-15 agonist, an IL-15 superagonist, or a combination thereof.

In some embodiments, the NK cell-stimulating agent is an IL-15 superagonist.

In some embodiments, the NK cell-stimulating agent is not dupilumab or IL-15.

In some embodiments, the NK cell-stimulating agent increases the NK cell level or function in the subject to a level above 97.5 percentile.

In some embodiments, the NK cell-stimulating agent is administered in an amount effective to prevent or ameliorate symptoms of the allergic disorder.

In some embodiments, ameliorating symptoms of the allergic disorder comprises: reducing redness and scaling (clinical score 0-5); reducing Numerical Rating scale (NRS) itch score; reducing Investigator Global Assessment (IGA) score; and/or reducing inflammatory, AD-associated serum biomarkers, TARC (CCL17), IL-4, or IL-13.

In some embodiments, the NK cell-stimulating agent is administered in an amount effective to ameliorate symptoms associated with atopic dermatitis (AD).

In some embodiments, ameliorating symptoms of atopic dermatitis (AD) comprises reducing erythema (redness), scaling, blood eosinophilia, serum IgE, or itch behavior (pruritus).

In some embodiments, the NK cell-stimulating agent is administered in an amount effective to improve histopathologic features selected from one or more of the group consisting of acanthosis (epidermal thickening), hyperkeratosis (stratum corneum thickening), spongiosis (epidermal edema), and mixed dermal lymphocyte and eosinophil infiltration.

In some embodiments, the NK cell-stimulating agent induces NK cell expansion in a dose-dependent manner.

In some embodiments, the NK cell-stimulating agent comprises an NK cell checkpoint inhibitor.

In some embodiments, the NK cell-stimulating agent comprises an IL-32 inhibiting agent, an IL-32α inhibiting agent, an IL-4 inhibiting agent, an IL-4 receptor α inhibiting agent, an IL-13 inhibiting agent, or an IL-13 receptor α inhibiting agent, or a combination thereof.

In some embodiments, the NK cell-stimulating agent comprises an IL-15 agonist, an IL-15 superagonist, or a combination thereof; and/or an IL-32a inhibiting agent, an IL-32 inhibiting agent, an IL-4 inhibiting agent, an IL-4 receptor α inhibiting agent, an IL-13 inhibiting agent, or an IL-13 receptor α inhibiting agent, or a combination thereof. In some embodiments, the IL-15 superagonist is selected from an IL-15:sIL-15Rα complex; a receptor-linker-IL-15 (RLI), a fusion polypeptide of IL-15 and IL-15Rα Sushi domain; ALT-803, a complex of IL-15 mutant IL-15N72D and a Sushi domain of IL-15Rα; or a combination thereof. In some embodiments, the IL-32 inhibiting agent is an anti-IL-32 mAb; the IL-32α inhibiting agent an anti-IL-32α mAb; the IL-4 inhibiting agent is an anti-IL-4 mAb; the IL-4 receptor α inhibiting agent is an anti-IL-4 receptor α mAb; the IL-13 inhibiting agent is an anti-L-13 mAb; or the IL-13 receptor α inhibiting agent is an anti-IL-13 mAb.

In some embodiments, the NK cell-stimulating agent is a bispecific monoclonal antibody capable of simultaneously enhancing IL-15 activity and reducing IL-32α activity, IL-32 activity, IL-4 activity, IL-4 receptor α activity, IL-13 activity, or IL-13 receptor α activity.

In some embodiments, the NK cell-stimulating agent is a monoclonal antibody or bispecific monoclonal antibody comprising one or more of the group consisting of: an IL-15 agonist, an IL-15 superagonist, an IL-32α inhibiting agent, an IL-32 inhibiting agent, an IL-4 inhibiting agent, an IL-4 receptor α inhibiting agent, an IL-13 inhibiting agent, or an IL-13 receptor α inhibiting agent, or a combination thereof.

In some embodiments, increasing the NK cell population comprises increasing total NK cell population.

In some embodiments, increasing the NK cell population comprises increasing mature CD56^{dim} NK cell levels.

In some embodiments, a therapeutically effective amount of an NK cell-stimulating agent increases NK cell function before administration of the NK cell-stimulating agent.

In some embodiments, NK cell levels or NK cell function is measured in a sample comprising blood, optionally, peripheral blood.

In some embodiments, the method further comprises administering a type 2 cytokine blockade therapy, optionally, dupilumab, to the subject.

Other objects and features will be in part apparent and in part pointed out hereinafter.

### DESCRIPTION OF THE DRAWINGS

Those of skill in the art will understand that the drawings, described below, are for illustrative purposes only. The drawings are not intended to limit the scope of the present teachings in any way.

FIG. 1. NK cell deficiency is a diagnostic feature of moderate-to-severe AD compared to non-AD and patients with chronic pruritus of unknown origin (CPUO). (A to D) Retrospective clinical laboratory values (number of cells/mm³ peripheral blood) of (A) CD4 T cells, (B) CD8 T cells, (C) B cells, and (D) NK cells in 25 moderate-to-severe AD and 363 non-AD control patient subjects. (E) Histogram of NK cells/mm³ from AD cohort, with the dashed line indicating the bottom 2.5th percentile of normal values for this laboratory test. (F) Receiver operating characteristic (ROC) curve of NK cell number as a diagnostic test for AD versus a non-AD control patient population of all-comers tested during the same time period (except oncology). An optimal sensitivity of 72% and a specificity of 75.9% were found at a cutoff of ≤95 NK cells/mm³. (G) ROC curve of NK cell number for AD versus CPUO cohorts (N=69 patients with CPUO), with a cutoff of ≤130 NK cells/mm³ providing optimal sensitivity (80%) and specificity (75%). AUC, area under the curve. ***P<0.001; NS, P>0.05 by Mann-Whitney U test.

FIG. 2. Patients with AD exhibit alterations in circulating NK cell subpopulations. (A) Sample collection and analysis schematic for CyTOF-based profiling of peripheral blood NK cells in six AD and five control subjects. (B) Representative viSNE plot colored by subpopulation identity. See Materials and Methods and FIG. 8 to FIG. 10 for additional gating information. tSNE, t-distributed stochastic neighbor embedding. (C) Marker signal intensity for select markers indicated above the plots on a representative AD sample. Blue lines indicate subpopulation gates. (D) Heatmap of median arcsine-normalized signal intensities for each viSNE-based subpopulation. (E) Frequency of cells in each subpopulation as a percentage of total NK cells. *P<0.05 by two-way ANOVA.

FIG. 3. Type 2 cytokine blockade rescues NK cell deficiency in human AD. (A) Schematic of anti-IL-4Rα mAb (monoclonal antibody; dupilumab) treatment monitoring. Dupilumab was administered every other week (q2w) subcutaneously (s.c.) for at least 3 months. (B) Investigator Global Assessment (IGA) score for each patient before and after treatment. Scoring is conducted on a six-point scale, in which 0=no disease and 5=very severe disease. N=6 patients with AD. **P<0.01 by Mann-Whitney U test. (C to E) Luminex ELISA measurements of plasma (C) thymus and activation-regulated cytokine (TARC; also known as CCL17), (D) IL-4, and (E) IL-13 in healthy controls (N=11) and patients with AD (N=10) before and after dupilumab treatment. *P<0.05 by unpaired, two-tailed t test. ^{†}P<0.05 and ^{††}P<0.01 by paired, two-tailed t test between pre- and posttreatment values for each individual. (F) Subpopulation frequencies as determined by CyTOF multidimensional analysis per patient before and after treatment (N=6 patients). *P<0.05 by paired two-tailed t test. ^{‡}P<0.05 when excluding patients with NK cells≥95 cells/mm³ (open circles) in a two-tailed, paired t test. (G) Number of NK cells (cells/mm³ blood) from clinical laboratory testing for total Lin⁻ (CD3, CD19) CD56⁺/CD16⁺ cells before and after treatment. *P<0.05 by paired, two-tailed t test.

FIG. 4. NK cells from patients with AD exhibit evidence of activation-induced cell death (AICD). (A) Schematic of CD56^{dim} NK cell isolation for RNA-seq (N=4 AD and 5 control individuals). (B) PCA of CD56^{dim} NK cells from AD and control individuals. (C) Volcano plot with significantly differentially expressed genes, with >2-fold change highlighted in red (up-regulated in AD) and blue (down-regulated in AD). (D) Broad categories of differentially expressed GO terms plotted by their enrichment scores (see Materials and Methods). (E) Enrichment scores of significantly differentially enriched (P<0.05) apoptosis-associated gene sets by GSEA. (F) Select apoptosis gene set-associated genes differentially expressed in AD versus control CD56^{dim} NK cells, represented as z-scored RPKM (reads per kilobase per million reads). (G) Frequency of CD56^{dim} NK cells that are cleaved caspase-3⁺ by flow cytometry from either control or AD PBMCs. N=5 healthy control and 5 patients with AD. (H) Schematic of AICD in vitro assay. (I) Percent change in the frequency of live CD56^{dim} NK cells of Lin⁻ (CD3, CD19, CD14, CD34) cells after 3 hours of in vitro CD16 ligation. *P<0.05 by unpaired, two-tailed t test. N=9 non-AD patient controls and 8 patients with AD. (J) GSEA enrichment plot of AD versus control gene expression compared to a previously published IL-2, IL-12, and IL-18 cytokine-induced gene set (40). FDR, false discovery rate. NES, normalized enrichment score, FWER, familywise-error rate. (K and L) Plasma (K) IL-12 and (L) IL-18 measured by Luminex multiplex ELISA in samples taken from 10 patients with AD before and at least 3 months after initiation of dupilumab treatment as well as from 11 healthy control donors. *P<0.05 with an unpaired t test, ^{†}P<0.05 with a paired t test between pre- and post-treatment values for each individual. (M) Frequency of cleaved caspase-3⁺ CD56^{dim} NK cells by flow cytometry from control PBMCs after overnight culture. ***P<0.001 by two-tailed t test. (N) Percent change from baseline of live CD56^{dim} NK cells 3 hours after CD16 ligation in control PBMCs preincubated with either medium alone or medium with IL-12 and IL-18 overnight. *P<0.05 by two-tailed t test.

FIG. 5. NK cells are enriched in lesional AD skin and limit type 2 inflammation. (A) Sample acquisition schematic for RNA-seq of lesional (L) and nonlesional (NL) skin biopsies of six patients with moderate-to-severe AD. (B) Select differentially expressed gene ontology (GO) terms (P<0.05) between lesional and nonlesional skin plotted by the enrichment score (see Materials and Methods). (C) Abundance (in arbitrary units) of activated NK cells imputed with CIBERSORT in lesional versus nonlesional patient skin biopsies (N=6 pairs of biopsies). *P<0.05 by paired, two-tailed t test. (D) Schematic of AD-like disease induction using MC903 or ethanol (EtOH, vehicle) application (n=4 to 5 mice per group). (E) Select differentially expressed GO terms (P<0.05) in murine AD-like skin compared to control skin plotted by their enrichment score (see Materials and Methods). (F) Flow cytometric analysis of murine AD-like (MC903) and control (EtOH) skin measuring the frequency of Lin⁻ (CD3, CD5, CD19, FcεRIα) NK1.1⁺ NK cells. (G) Diagram of the murine AD model for the comparison of WT and Il15^{-/-} mice. (H) Frequency of Lin⁻ (CD3, CD11c, CD19) NK1.1⁺ cells in the lesional skin of WT and Il15^{-/-} mice measured on day 12 by flow cytometry. (I) Gene expression of Ifng by quantitative reverse transcription PCR in lesional skin on day 12. (J and K) Frequency on day 12 by flow cytometry of (J) Lin⁻ (CD3, CD5, CD11b, CD11c, CD19, NK1.1) KLRG1⁺ ST2⁺ ILC2s and (K) Siglec-F⁺ eosinophils in lesional skin. *P<0.05 by unpaired two-tailed t test.

FIG. 6. IL-15 superagonism boosts NK cells and alleviates AD-like disease in mice. (A) Diagram of IL-15 superagonist (SA) administered to mice. (B) Schematic of IL-15SA or IgG1 isotype administration during AD model. (C and D) Number of Lin⁻ (CD3, CD11c, CD19) NK1.1⁺ CD49b⁺NK cells on experimental day 8 in the (C) blood and (D) skin. (E and F) Numbers of (E) ILC2s and (F) eosinophils in AD-like skin on experimental day 8. *P<0.05, **P<0.01, and ***P<0.001 by unpaired two-tailed t test. (G) Representative images of lesional skin on experimental day 12 of IgG1 isotype- and IL-15SA-treated mice. (H) Clinical scores and (I) ear thickness (percent change from baseline) of isotype- and IL-15SA-treated mice during the course of treatment. **P<0.01 by two-way ANOVA. (J) Representative H&E histopathology images of ear skin on experimental day 12. Scale bars, 100 μm. Arrowheads indicate inflammatory vasodilation. (K) Histopathology score of H&E sections on experimental day 12. *P<0.05 by unpaired, two-tailed t test. (L) Treatment schematic of IL-15SA or isotype treatment and AD-like disease induction in Cd8^{-/-} mice. (M) Ear thickness (percent change from baseline) and (N) clinical scores of isotype control- and IL-15SA-treated Cd8^{-/-} mice. *P<0.05 by two-way ANOVA. (O) Treatment schematic of IL-15SA or isotype treatment and AD-like disease induction in NK cell-deficient Ncr1-iCre Rosa-DTA (Ncr1^{DTA}) mice. (P) Ear thickness (percent change from baseline) and (Q) clinical scores of isotype- and IL-15SA-treated Ncr1^{DTA} mice. NS, P>0.05 by two-way ANOVA.

FIG. 7. Clinical lymphocyte laboratory results from patients with AD. (A) Representative flow cytometry plots from retrospective analysis of clinical laboratory results. (B-E) Frequency of CD45⁺ cells in retrospective analysis of (B) CD4 T cells, (C) CD8 T cells, (D) B cells, and (E) NK cells. **p<0.01 and NS p≥0.05 by Mann-Whitney U test. Notches in the box and whisker plots represent the 25th-75th percentiles, with the median line and whiskers marking the 1.5 inter-quartile range.

FIG. 8. Signal intensities and gating for phenotypic analysis of human peripheral blood NK cells by CyTOF. (A) tSNE plots of viSNE analysis and manual gating strategies from a representative patient with AD. tSNE plots are colored by signal intensity for each of the indicated markers. (B) Gating strategy for populations shown in FIG. 2-3.

FIG. 9. Density plots of viSNE analysis of CyTOF data for Lin⁻ CD56⁺ cells by subject. Density heatmaps of CD45⁺ CD3⁻ CD19⁻ CD14⁻ CD56⁺ events from CyTOF viSNE analysis for each individual control and AD donor. AD donors (except AD005) received dupilumab treatment and have both a pre-treatment and post-treatment sample.

FIG. 10. Detailed signal intensities of CyTOF viSNE populations. (A) A comparison of CyTOF signal intensities of maturation markers between mature and immature CD56dim NK cells across all groups (N=11). (B-D) A comparison of CyTOF signal intensities between NCR⁻ cells, non-NK cell population number 2, mature CD56dim NK cells, and immature CD56dim NK cells separated by (B) activating receptors, (C) NK cell transcription factors and lytic proteins, and (D) maturation and activation markers in healthy and patient controls (N=5). *p<0.05, **p<0.01, ***p<0.001 by One-Way ANOVA.

FIG. 11. RNA-seq of human CD56^{dim} NK cells. (A) Gating strategy for sort-purification of human CD56^{dim} NK cells. (B) Heatmaps of differentially expressed genes with GO terms, represented as z-scored RPKM from 5 non-AD patient controls and 4 patients with AD. Genes are grouped by the GO term categories shown in FIG. 4D.

FIG. 12. Gating strategies for in vitro assays on CD56^{dim} NK cells. (A) Gating strategy for a representative sample for detection of intracellular cleaved caspase-3 in CD56^{dim} NK cells by flow cytometry. (B) Representative histogram of cleaved caspase-3 staining in CD56^{dim} NK cells from AD and non-AD patient controls. (C) Frequency of CD56^{dim} NK cells as a percentage of Lin⁻ (CD3, CD19, CD34, CD14)⁻ cells in PBMC from non-AD patient controls and patients with AD after overnight culture in basal media (N=9 per group). **p<0.01 by two-tailed unpaired t test. (D) Frequency of dead CD56^{dim} NK cells by 7-AAD staining in control and AD PBMC after overnight culture in basal media (N=9 per group). NS p>0.05 by two-tailed unpaired t test. (E) Gating strategy for identification of CD56^{dim} NK cells in a representative sample in anti-CD16 ligation experiments. MFI=median fluorescence intensity.

FIG. 13. AD NK cells demonstrate evidence of AICD. (A) Significantly differentially expressed genes (DESeq2 adjusted p<0.05) from AD versus control CD56^{dim} NK cells that overlap with genes also identified by Smith et al. (40) to be differentially expressed by cytokine-stimulated NK cells, represented as z-scored RPKMs. (B) Schematic of total NK cell purification by negative bead selection for in vitro CD16 stimulation assay. (C) Frequency of CD56^{dim} NK cells that are positive for cleaved caspase-3 following CD16 stimulation in either media alone or IL-12/18 pre-incubation overnight (n=6 technical replicates). **p<0.01 by two-tailed Student's t test. (D) Number of CD56^{dim} NK cells following stimulation with different concentrations of anti-CD16-coated microbeads after IL-12/18 pre-incubation (n=3 technical replicates). (E) Expression of FCGR3A (CD16) in sort-purified CD56^{dim} NK cells from control and AD donors. *p<0.05 by two-tailed unpaired t test (N=5 non-AD patient controls and 4 patients with AD). (F) viSNE plot from a representative AD donor showing the bulk CD56^{dim} NK cell gating strategy. (G) CD16 mean fluorescence intensity after arcsine transformation and normalization of CD56^{dim} NK cells from CyTOF experiments on PBMC from 5 healthy and patient controls and 6 patients with AD. p value from two-tailed unpaired t test.

FIG. 14. NK cell enrichment in AD skin of mice and humans. (A) PCA of sort-purified CD56dim NK cells from 4 patients with AD and 5 healthy or non-AD patient control donors. (B) Differentially expressed genes with NK cell-associated GO term annotations. Heatmap represents z-scored RPKM. (C) Schematic of MC903-induced AD-like disease in mice. (D) NK cell numbers in lesional AD-like skin of mice on experimental day 12 of MC903 treatment by flow cytometry (n=3 EtOH-treated and 5 MC903-treated mice). (E) Frequency of NK1.1+ NK cells as a percentage of Lin⁻ (CD3, CD5, CD19, FcεRIα) cells in peripheral blood of control (EtOH, n=12) and AD-like (MC903, n=13) mice on experimental day 12. Box and whisker plot represents the 25^{th}-75^{th} percentiles, with a median line and whiskers indicating the minimum and maximum values. *p<0.05 by two-tailed unpaired t test.

FIG. 15. Gating strategy for murine skin immune cell populations. (A) Representative gating for identification of ILC2s and NK cells in WT and Il15^{-/-} mice. (B) Representative gating for identification of eosinophils in murine skin.

FIG. 16. NK cell depletion results in enhanced ILC2 responses. (A) Schematic of treatment of WT and Il15^{-/-}, mice with MC903 to induce AD-like disease (n=5 mice per group). (B) NK cell frequency in the skin draining lymph nodes (sdLN) on day 12 of MC903 treatment. (C) Representative FACS plots of ILC2s in the sdLN of WT and Il15^{-/-}, mice. (D) Frequency of ILC2s in Lin⁻ (CD3, CD5, CD11b, CD11c, NK1.1) CD127⁺ cells in the sdLN of WT and Il15^{-/-} mice. (E) Frequency of ILC2s in sdLN of naïve Il15^{-/-} mice compared to WT controls by flow cytometry. (F) Schematic of NK cell depletion with 100 μg anti-NK1.1 (PK136, Bio-X-cell) or isotype control (C1.18.4, Bio-X-cell) antibodies administered i.p. on indicated days concomitant with MC903 treatment for induction of AD-like disease in WT mice (n=5 mice per group). (G) Frequency of NK cells in sdLN of anti-NK1.1 and isotype-treated mice on day 12 of MC903 treatment. (H) Representative flow plots of ILC2s in the sdLN of WT mice. (I) Frequency of ILC2s in Lin⁻ (CD3, CD5, CD11b, CD11c, NK1.1) cells in sdLN of WT mice on day 12 of MC903 treatment. (J) Treatment regimen of NK cell depletion with 100 μg anti-NK1.1 (PK136, Bio-X-cell) or isotype control (C1.18.4, Bio-X-cell) antibodies administered i.p. on indicated days in Rag1^{-/-} mice (n=5 mice per group). (K) Frequency of NK cells in sdLN of Rag1^{-/-} on day 12 of MC903 treatment. (L) Representative flow plots of ILC2s in Rag1^{-/-}, skin sdLN on day 12. (M) Frequency of ILC2s in Lin⁻ (CD3, CD5, CD11b, CD11c, CD19, NK1.1) cells in sdLN of Rag1^{-/-} mice on day 12 of MC903 treatment. (N) Ear thickness as a percent change from baseline during MC903 treatment of WT mice given 100 μg anti-NK1.1 (PK136) or 100 μg isotype control (C1.18.4) on experimental days −2 and 7. n=11-13 mice per group. (O) Ear thickness as a percent change from baseline during MC903 treatment of WT and Il15^{-/-}, mice. n=7-9 mice per group. p values shown are from a Two-way ANOVA. **p<0.01, ***p<0.001 by unpaired, two-tailed t test.

FIG. 17. IL-15 SA induces a dose-dependent NK cell expansion and ameliorates AD-like disease at later time points. (A) The amount of free recombinant murine (rm) IL-15 measured by ELISA in superagonist (SA) complexes with molar ratios of 3.4:1 and 1.7:1 as well as IL-15-only and IL-15Rα-Fc-only control solutions. (B) Frequency of NK1.1⁺ CD49b⁺ NK cells in peripheral blood of mice (n=2) following 4 days of different doses of IL-15 SA treatment as a percentage of Lin⁻ (CD3 CD5 CD19 FcεRIα) cells. (C) Frequency of NK1.1⁺ CD49b⁺ NK cells in ear skin of mice (n=2) following 4 days of different doses of IL-15 SA treatment as a percentage of Lin⁻ (CD3 CD5 CD19 FcεRIα) cells. (D) Treatment schematic for IL-15 SA and IgG1 isotype control. (E) Daily percent change in ear thickness measurements over baseline. n=5 mice per group. (F) Daily clinical scoring of redness and scaling. n=5 mice per group. *p<0.05, **p<0.01 by two-way ANOVA.

FIG. 18. Ncr1-DTA mice lack NK cells but have intact CD8 T cells. (A) Representative flow cytometry plot of NK cells in naïve WT and Ncr1^{DTA} mice. (B-C) Frequency of blood (B) and skin (C) NK cells in naïve WT (n=6) and Ncr1^{DTA} mice (n=6). (D) Representative flow cytometry plot of NK cells in naïve WT and Ncr1^{DTA} mice. (E-F) Frequency of blood (E) and skin (F) NK cells in naïve WT and Ncr1-DTA mice. n=6 mice per group. *p<0.05, ****p<0.0001, and NS p≥0.05 by two-tailed unpaired t test.

FIG. 19 is a schematic depiction of three forms of IL-15 agonists. a) IL-15:sIL-15Rα complex. b) receptor-linker-IL-15 (RLI), a fusion polypeptide of IL-15 and IL-15Rα Sushi domain. c) ALT-803, a complex of IL-15 mutant IL-15N72D with the Sushi domain of IL-15Rα. (Wu, 2013).

FIG. 20 is a table showing treatment design for days 0 through 12 and a series of photos of ears on day 12.

FIG. 21 are graphs depicting caliper measurement of ear thickness and clinical scores for days 0 through 12.

FIG. 22 is a graph depicting the combined clinical score for days 0 through 12.

### DETAILED DESCRIPTION OF THE INVENTION

The present disclosure is based, at least in part, on the discovery that natural killer (NK) cells are deficient in atopic dermatitis (AD) and that immunomodulators that stimulate NK cells relatively selectively boost NK cell function and will improve AD and other related allergic disorders (e.g., eczema, food allergy, asthma, eosinophilic esophagitis or eosinophilic gastrointestinal disorders, chronic rhinosinusitis). It was also discovered that IL-15 agonists known to boost NK cell function result in attenuation of AD-like disease in an accepted model of AD disease.

Described herein, is new data describing how NK cells are deficient in human AD. It is further shown that NK cells are required for control of type 2 inflammation that drives AD pathogenesis. Taken together, these findings indicate that immunomodulators that stimulate NK cells relatively selectively boost NK cell function and will improve AD and other related allergic disorders (e.g., eczema, food allergy, asthma, eosinophilic esophagitis or eosinophilic gastrointestinal disorders, chronic rhinosinusitis). It has also been previously shown that studies of cancer immunotherapy have looked at enhanced cytokine production (enhanced NK cell function) in a patient's blood following IL-15SA treatment.

Recombinant IL-15 (i.e., simply replacing IL-15) has been ineffective almost universally as a therapeutic. As described herein, NK cell stimulation (e.g., by IL-15 super agonism) is a synthetic strategy that is not equivalent to a simple replacement of IL-15 levels to normal levels. IL-15 super agonism results in supraphysiologic activity over and beyond simply restoring IL-15 levels to normal levels.

Validations of the findings described herein were performed and described later in Mobus et al., Journal of Allergy and Clinical Immunology, In Press, Available online 29 Jun. 2020.

Allergic Disorders

As described herein are methods of treating atopic dermatitis and other related allergic disorders, such as allergic disorders that are characterized or associated with NK cells. An allergic disorder associated with NK cells can be a disorder in which the subject has depleted NK cells (e.g., level, function) or a boost in NK cells ameliorates symptoms of the allergic disorder (e.g., increasing compared to baseline, supraphysiologic levels). An allergic disorder can be an allergic disorder related to atopic dermatitis (e.g., an atopic dermatitis-related allergic disorder, allergic disorder associated with NK cell deficiency) such as eczema, food allergy, asthma, eosinophilic esophagitis or eosinophilic gastrointestinal disorders, allergic rhinitis, and/or chronic rhinosinusitis. An allergic disorder can be a disorder in which a boost in NK cells ameliorates symptoms, even if there is not an NK cell deficiency. An allergic disorder can be an allergic disorder characterized by NK cell depletion or can be an allergic disorder characterized by amelioration of symptoms upon NK cell boosting (an NK cell-associated allergic disorder). As described herein, the allergic disorder can be a type 2 inflammatory condition, such as AD, asthma, and food allergy that are characterized by elevated IgE, eosinophilia, and a predisposition for allergen sensitization across barrier surfaces. As described herein, the subject having an allergic disorder can be a subject with atopy or atopic syndrome, the genetic tendency to develop allergic diseases such as allergic rhinitis, asthma, and atopic dermatitis (eczema). Atopy is typically associated with heightened immune responses to common allergens, especially inhaled allergens, and food allergens. Atopy is the tendency to produce an exaggerated IgE immune response to otherwise harmless environmental substances, while an allergic disease can be defined as the clinical manifestation of this inappropriate IgE immune response.

NK Cell-Stimulating Agent

As described herein, it was discovered that NK cells were depleted in AD. It was also discovered that IL-15 agonists can be used as an NK cell-stimulating agent for the treatment of AD. It is believed that an NK cell-stimulating agent can also be used for the treatment of other diseases associated with NK cell depletion. The NK cell-stimulating agent can also be a bispecific antibody that can mobilize two different cytokines at once. As such, the agent can be a drug that blocks IL-32α, IL-4, IL-13, IL-4 receptor α, and/or IL-13 receptor α while simultaneously boosting IL-15 activity (e.g., via an agonist, a superagonist approach, or another method). Measuring NK cell levels and function can be achieved through methods known in the art (see e.g., Orange et al. J Allergy Clin Immunol. 2013 September; 132(3): 515-526).

An NK cell-stimulating agent can be an agent or therapeutic that activates NK cells, boosts, or restores NK cell function (e.g., flow cytometry, in vitro killing assays, detecting levels of NK GO terms), or increases a quantity of NK cells (e.g., monitoring levels of NK cells in blood) in a subject or the tissue of a subject. An NK cell-stimulating agent can be an IL-15 agonist or IL-15 superagonist. NK cell-stimulating agents can be used to treat allergic disorders (e.g., atopic dermatitis, eczema, etc.). It is believed that all therapies for treating atopic dermatitis currently available or in development involve shutting down type 2 inflammation. It is believed that IL-15SAs may also shut down type 2 inflammation via T cells. However, patients with atopic dermatitis have a deficiency in type 1 immunity and NK cells. The disclosed technology would employ IL-15 superagonists (e.g., such as those that boost NK cell stimulation, increasing NK cell numbers, increasing NK cell function) to treat atopic dermatitis and other allergic disorders in an entirely novel manner.

An IL-15 agonist can be an IL-15 superagonist, such as an IL-15 superagonist complex or a functional variant thereof, including mutants with IL-15 agonist or superagonist function. IL-15 agonists, superagonists, and uses thereof are well known; see e.g., Wu IL-15 Agonists: The Cancer Cure Cytokine. J Mol Genet Med 7:85; Felices et al. Gynecol Oncol. 2017 June; 145(3):453-461; Zhu et al. J Immunol 2009; 183:3598-3607; Kim et al. Oncotarget. 2016 Mar. 29; 7(13):16130-45. Except as otherwise noted herein, therefore, the IL-15 agonists (including functional variants and uses thereof) of the present disclosure can be carried out in accordance with such processes.

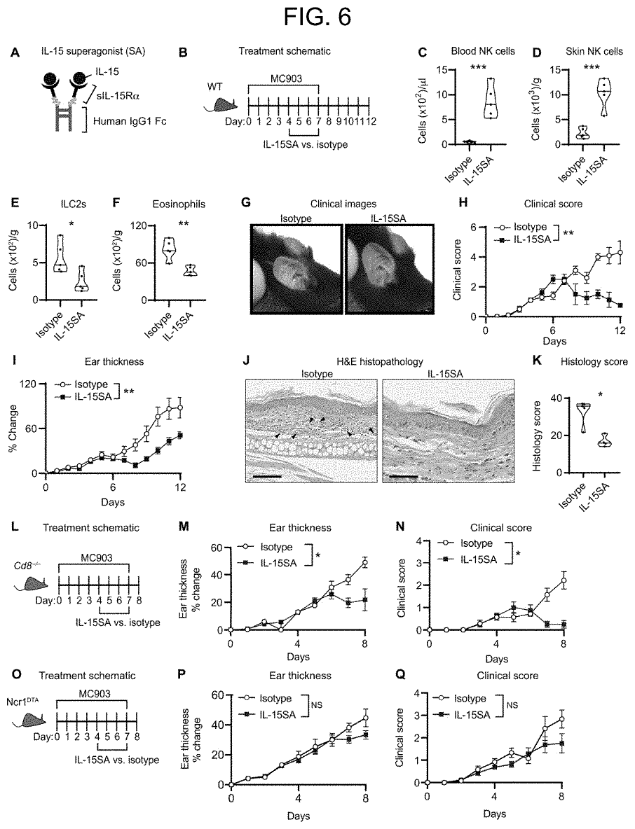

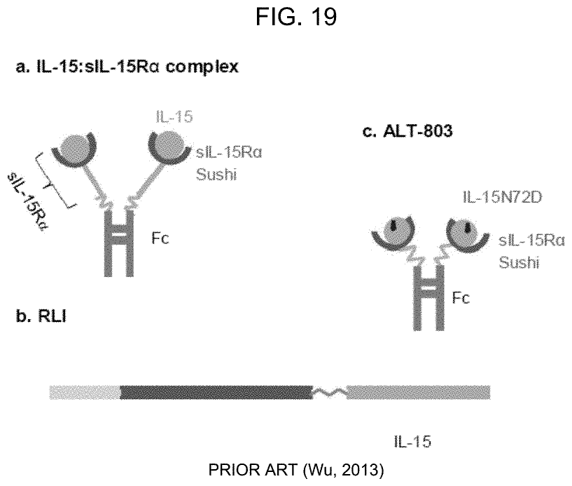

IL-15 agonists or IL-15 superagonists can be an IL-15:sIL-15Rα complex, ALT-803 (aka N-803), or RLI (see e.g., FIG. 6A, FIG. 19). Other IL-15 superagonists, such as hetIL-15 and NKTR-255 can be used as well (see e.g., Knudson et al. Expert Opin. On Biol. Ther. 2020 (7), incorporated herein by reference).

Examples of IL-15 superagonists in clinical development.

| | | | | Clinical |
|---|---|---|---|---|
| | | Clinical | | Development |
| Agent | Description | Designation | Company | Status |
| Heterodimeric | Secreted IL- | NIZ985 | Novartis | Phase I (1 |
| IL-15 | 15/IL-15Rα | | | active trial) |
| (hetIL-15) | complex | | | |
| Receptor-linked | Fusion protein of | SO-C101 | SOTIO/Cytune | Phase I (1 |
| IL-15 (RLI) | rIL-15 linked to | | Pharma | active trial) |
| | IL-15Rα sushi | | | |
| | (cytokine-binding) | | | |
| | domain via a 20-amino | | | |
| | acid flexible | | | |
| | linker | | | |
| NKTR-255 | IL-15Rα- | NKTR-255 | Nektar | Phase I (1 |
| | dependent | | Therapeutics | active trial) |
| | polymer- | | | |
| | conjugated | | | |
| | recombinant | | | |
| | human IL-15 | | | |
| | (rhIL-15) agonist | | | |
| N-803 (formerly | Fusion protein | N-803 | ImmunityBio | Phase I/II (23 |
| ALT-803) | consisting of a | (ALT-803) | | active trials) |
| | mutated | | | |
| | superagonist IL- | | | |
| | 15 (N72D) bound | | | |
| | to IL-15Rα sushi | | | |
| | domain linked to | | | |
| | IgG1-Fc domain | | | |

Examples of IL-15 superagonists in clinical trials

| | Combination | | | Trial |
|---|---|---|---|---|
| Agent | Agent | Cancer Type | Phase | Identifier |
| NIZ985 | PDR001 | Metastatic and | Phase | NCT02452268 |
| | | advanced solid | I/Ib | |
| | | tumors | | |
| SO-C101 | — | Metastatic and | Phase | SC103 |
| | | advanced solid | I/Ib | |
| | | tumors | | |
| NKTR-255 | Daratumumab | Multiple myeloma, | Phase | NCT04136756 |
| | | NHL | I | |
| N-803 | — | Relapsed or | Phase | NCT02099539 |
| | | refractory multiple | I/II | |
| | | myeloma | | |
| | — | AML, MDS (post- | Phase | NCT02989844 |
| | | allogenic HCT) | II | |
| | Intravesical BCG | High grade NMIBC | Phase | NCT03022825 |
| | | (BCG | II | |
| | | unresponsive) | | |
| | Intravesical BCG | NMIBC (BCG | Phase | NCT02138734 |
| | | naïve) | Ib/IIb | |
| | Nivolumab | Advanced or | Phase | NCT02523469 |
| | | metastatic NSCLC | Ib/II | |
| | | (PD-1/PD-L1 | | |
| | | checkpoint inhibitor | | |
| | | refractory or naive) | | |
| | Pembrolizumab, | Advanced solid | Phase | NCT03228667 |
| | nivolumab, | carcinomas | IIb | |
| | atezolizumab, | (progression | | |
| | avelumab | following initial | | |
| | | response to PD- | | |
| | | 1/PD-L1 checkpoint | | |
| | | inhibitors) | | |
| | M7824, MVA-BN- | Prostate, advanced | Phase | NCT03493945 |
| | Brachyury, | solid tumors | I/II | |
| | epacadostat | | | |
| | Rituximab | Relapsed or | Phase | NCT02384954 |
| | | refractory iNHL | I/II | |
| | aNK(NK-92) | Stage III/IV MCC | Phase | NCT02465957 |
| | | | II | |
| | Avelumab, haNK | MCC (PD-1/PD-L1 | Phase | NCT03853317 |
| | | checkpoint inhibitor | II | |
| | | refractory) | | |
| | Pembrolizumab | Stage III/IV NSCLC | Phase | NCT03520686 |
| | | | II | |

Other NK cell-stimulating agents (e.g., such as anti-cancer drugs), variants having NK cell-stimulating function, and uses thereof are well known; see e.g., Cifaldi et al. Boosting Natural Killer Cell-Based Immunotherapy with Anticancer Drugs: a Perspective, Trends in Molecular Medicine 2017 23(12); Romagne et al. Natural killer cell-based therapies, F1000 Med Rep. 2011 3(9).

An NK cell-stimulating agent can also be any agent or therapeutic that blocks or reduces the function of inhibitors of NK cell function. Because IL-32α directly acts on NK cells to make them less responsive to IL-15 and thus less activated (Gorvel et al., J Immunol. 2017 Aug. 15; 199(4):1290-1300), IL-32α blockade with a monoclonal antibody can act alone as an NK cell-stimulating agent or can enhance the activity of an IL-15 superagonist. As such, an NK cell-stimulating agent can be an IL-32α inhibiting agent, alone or in combination with an IL-15 superagonist to boost IL-15. Similarly, an NK cell-stimulating agent can be an IL-32 inhibiting agent, an IL-4 inhibiting agent, an IL-4 receptor α inhibiting agent, an IL-13 inhibiting agent, or an IL-13 receptor α inhibiting agent, alone or in combination with an IL-15 superagonist to boost IL-15. As an example, the NK cell-stimulating agent can comprise an anti-IL-32 mAb, an anti-IL-32α mAb, an anti-IL-4 mAb, an anti-IL-4 receptor α mAb, an anti-L-13 mAb, or an anti-IL-13 mAb, functional variant thereof, or a combination thereof.

Other methods to stimulate NK cells are methods that can comprise blocking inhibitors of NK cell function, which enhances the activity of IL-15 agonists or IL-15 superagonists. Simultaneously reducing the activity of inhibitors of NK cells function and enhancing the activity of IL-15 can be a synergistic approach for stimulating NK cell activity and treating topical dermatitis and other allergic disorders. This method can employ administration of a bispecific monoclonal antibody capable of simultaneously inhibiting IL-32α, IL-32, IL-4, IL-4 receptor α, IL-13, or IL-13 receptor α and activating IL-15, with, for example, an IL-15 superagonist.

As another example, an NK cell-stimulating agent can be an NK cell agonist or NK cell checkpoint inhibitor, such as those used in cancer immunotherapy. NK cell agonists and NK cell checkpoint inhibitors have proven highly effective in both expanding host NK cells and boosting their function.

An NK cell-stimulating agent can be used to boost NK cells to supraphysiologic levels which can promote their regulatory function and ameliorate disease. An example of supraphysiological levels can be an NK cell level>97.5 percentile. Normal ranges vary from lab to lab, thus percentiles can be applied to any lab. The normal range is the central 95 percentiles (2.5% to 97.5%). As another example, supraphysiological levels can be a level of over 130 NK cells/mm³. As another example, supraphysiological levels can be at least a 2-fold increase in NK cells following IL-15 SA therapy compared to baseline NK cells/mm³ before treatment.

An NK cell-stimulating agent can be administered to a subject having between 0 and 100^{th} percentile of NK cells (level or function, e.g., as measured by flow cytometry or in vitro killing assays). For example, the subject can have NK cells in the range of percentiles (%) of about 1%; about 2%; about 3%; about 4%; about 5%; about 6%; about 7%; about 8%; about 9%; about 10%; about 11%; about 12%; about 13%; about 14%; about 15%; about 16%; about 17%; about 18%; about 19%; about 20%; about 21%; about 22%; about 23%; about 24%; about 25%; about 26%; about 27%; about 28%; about 29%; about 30%; about 31%; about 32%; about 33%; about 34%; about 35%; about 36%; about 37%; about 38%; about 39%; about 40%; about 41%; about 42%; about 43%; about 44%; about 45%; about 46%; about 47%; about 48%; about 49%; about 50%; about 51%; about 52%; about 53%; about 54%; about 55%; about 56%; about 57%; about 58%; about 59%; about 60%; about 61%; about 62%; about 63%; about 64%; about 65%; about 66%; about 67%; about 68%; about 69%; about 70%; about 71%; about 72%; about 73%; about 74%; about 75%; about 76%; about 77%; about 78%; about 79%; about 80%; about 81%; about 82%; about 83%; about 84%; about 85%; about 86%; about 87%; about 88%; about 89%; about 90%; about 91%; about 92%; about 93%; about 94%; about 95%; about 96%; about 97%; about 98%; about 99%; or about 100%. Recitation of each of these discrete values is understood to include ranges between each value. Recitation of each range is understood to include discrete values within the range.

An NK cell-stimulating agent can be used to increase the number of NK cells in a subject (e.g., in blood). The number of NK cells in a subject can be between about 0 and about 1000 cells/mm³ of blood. For example, the number of NK cells in a subject can be about 1 cells/mm³; about 10 cells/mm³; about 20 cells/mm³; about 30 cells/mm³; about 40 cells/mm³; about 50 cells/mm³; about 60 cells/mm³; about 70 cells/mm³; about 80 cells/mm³; about 90 cells/mm³ about 100 cells/mm³; about 110 cells/mm³; about 120 cells/mm³; about 130 cells/mm³; about 140 cells/mm³; about 150 cells/mm³; about 160 cells/mm³ about 170 cells/mm³; about 180 cells/mm³; about 190 cells/mm³; about 200 cells/mm³; about 210 cells/mm³; about 220 cells/mm³; about 230 cells/mm³ about 240 cells/mm³; about 250 cells/mm³; about 260 cells/mm³; about 270 cells/mm³; about 280 cells/mm³; about 290 cells/mm³; about 300 cells/mm³ about 310 cells/mm³; about 320 cells/mm³; about 330 cells/mm³; about 340 cells/mm³; about 350 cells/mm³; about 360 cells/mm³; about 370 cells/mm³ about 380 cells/mm³; about 390 cells/mm³; about 400 cells/mm³; about 410 cells/mm³; about 420 cells/mm³; about 430 cells/mm³; about 440 cells/mm³ about 450 cells/mm³; about 460 cells/mm³; about 470 cells/mm³; about 480 cells/mm³; about 490 cells/mm³; about 500 cells/mm³; about 510 cells/mm³ about 520 cells/mm³; about 530 cells/mm³; about 540 cells/mm³; about 550 cells/mm³; about 560 cells/mm³; about 570 cells/mm³; about 580 cells/mm³ about 590 cells/mm³; about 600 cells/mm³; about 610 cells/mm³; about 620 cells/mm³; about 630 cells/mm³; about 640 cells/mm³; about 650 cells/mm³ about 660 cells/mm³; about 670 cells/mm³; about 680 cells/mm³; about 690 cells/mm³; about 700 cells/mm³; about 710 cells/mm³; about 720 cells/mm³ about 730 cells/mm³; about 740 cells/mm³; about 750 cells/mm³; about 760 cells/mm³; about 770 cells/mm³; about 780 cells/mm³; about 790 cells/mm³ about 800 cells/mm³; about 810 cells/mm³; about 820 cells/mm³; about 830 cells/mm³; about 840 cells/mm³; about 850 cells/mm³; about 860 cells/mm³ about 870 cells/mm³; about 880 cells/mm³; about 890 cells/mm³; about 900 cells/mm³; about 910 cells/mm³; about 920 cells/mm³; about 930 cells/mm³ about 940 cells/mm³; about 950 cells/mm³; about 960 cells/mm³; about 970 cells/mm³; about 980 cells/mm³; about 990 cells/mm³; or about 1000 cells/mm³. Recitation of each of these discrete values is understood to include ranges between each value. Recitation of each range is understood to include discrete values within the range.

An NK cell-stimulating agent can increase the amount of NK cells in a subject between more than 1-fold and at least about 2-fold. For example, the NK cell stimulating agent can increase the NK cells in a subject by about or by at least about 1%; about 2%; about 3%; about 4%; about 5%; about 6%; about 7%; about 8%; about 9%; about 10%; about 11%; about 12%; about 13%; about 14%; about 15%; about 16%; about 17%; about 18%; about 19%; about 20%; about 21%; about 22%; about 23%; about 24%; about 25%; about 26%; about 27%; about 28%; about 29%; about 30%; about 31%; about 32%; about 33%; about 34%; about 35%; about 36%; about 37%; about 38%; about 39%; about 40%; about 41%; about 42%; about 43%; about 44%; about 45%; about 46%; about 47%; about 48%; about 49%; about 50%; about 51%; about 52%; about 53%; about 54%; about 55%; about 56%; about 57%; about 58%; about 59%; about 60%; about 61%; about 62%; about 63%; about 64%; about 65%; about 66%; about 67%; about 68%; about 69%; about 70%; about 71%; about 72%; about 73%; about 74%; about 75%; about 76%; about 77%; about 78%; about 79%; about 80%; about 81%; about 82%; about 83%; about 84%; about 85%; about 86%; about 87%; about 88%; about 89%; about 90%; about 91%; about 92%; about 93%; about 94%; about 95%; about 96%; about 97%; about 98%; about 99%; or about 100% or more.

Formulation

The agents and compositions described herein can be formulated by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such formulations will contain a therapeutically effective amount of a biologically active agent described herein, which can be in purified form, together with a suitable amount of carrier so as to provide the form for proper administration to the subject.

The term “formulation” refers to preparing a drug in a form suitable for administration to a subject, such as a human. Thus, a “formulation” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc., Rockville, Md., 2005 (“USP/NF”), or a more recent edition, and the components listed in the continuously updated Inactive Ingredient Search online database of the FDA. Other useful components that are not described in the USP/NF, etc. may also be used.

The term “pharmaceutically acceptable excipient,” as used herein, can include any and all solvents, dispersion media, coatings, antibacterial and antifungal agents, isotonic, or absorption delaying agents. The use of such media and agents for pharmaceutically active substances is well known in the art (see generally Remington's Pharmaceutical Sciences (A. R. Gennaro, Ed.), 21st edition, ISBN: 0781746736 (2005)). Except insofar as any conventional media or agent is incompatible with an active ingredient, its use in the therapeutic compositions is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The formulation should suit the mode of administration. The agents of use with the current disclosure can be formulated by known methods for administration to a subject using several routes which include, but are not limited to, parenteral, pulmonary, oral, topical, intradermal, intranasal, inhalation (e.g., in an aerosol), implanted, intramuscular, intraperitoneal, intravenous, subcutaneous, epidural, ophthalmic, transdermal, buccal, and rectal. The individual agents may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic, or other physical forces.

Controlled-release (or sustained-release) preparations may be formulated to extend the activity of the agent(s) and reduce dosage frequency. Controlled-release preparations can also be used to affect the time of onset of action or other characteristics, such as blood levels of the agent and, consequently, affect the occurrence of side effects. Controlled-release preparations may be designed to initially release an amount of an agent(s) that produces the desired therapeutic effect, and gradually and continually release other amounts of the agent to maintain the level of therapeutic effect over an extended period of time. In order to maintain a near-constant level of an agent in the body, the agent can be released from the dosage form at a rate that will replace the amount of agent being metabolized or excreted from the body. The controlled-release of an agent may be stimulated by various inducers, e.g., change in pH, change in temperature, enzymes, water, or other physiological conditions or molecules.

Agents or compositions described herein can also be used in combination with other therapeutic modalities, as described further below. Thus, in addition to the therapies described herein, one may also provide to the subject other therapies known to be efficacious for the prevention or treatment of the disease, disorder, or condition.

Therapeutic Methods

Also provided is a process of treating or preventing an allergic disorder in a subject in need of administration of a therapeutically effective amount of an NK cell-stimulating agent (e.g., an IL-15 superagonist), so as to inhibit symptoms of an allergic disorder, slow the progress of an allergic disorder, limit the development of an allergic disorder, prevent symptoms of an allergic disorder, stimulate NK cells, boost NK cell function, increase an amount or a quantity of NK cells, or improve symptoms of atopic dermatitis or improve symptoms of other allergic disorders.

Improvement or amelioration of symptoms can include a clinical reduction in redness (e.g., using a clinical score 0-5; 0=none, 5=severe); a clinical reduction in scaling (e.g., using a clinical score 0-5; 0=none, 5=severe); a reduction in numerical rating scale (NRS) itch score (the NRS is comprised of one item and represents the numbers 0 (“no itch”) to 10 (“worst imaginable itch”), wherein subjects are asked to rate the intensity of their itch using this scale); improvement) or reduction) in Investigator Global Assessment (IGA) score (the 5-point IGA is a validated measure of disease severity and provides a clinically meaningful measure of success for psoriasis treatment studies); and/or reduction of inflammatory, AD-associated serum biomarkers, such as TARC (CCL17), IL-4, and IL-13. As another example, improvement or amelioration of symptoms can include improving scores, such as keratin thickness, epidermal thickness, epidermal spongiosis (e.g., 0 to 3 rating), microabscesses (e.g., 0 to 3 rating), vascular area (e.g., sum per treated side/1000), or inflammatory infiltrate (e.g., 0 to 10 rating). As another example, improvement or amelioration of symptoms can include improvement or amelioration of erythema (redness), scaling, blood eosinophilia, serum IgE elevation, itch behavior (pruritus), and histopathologic features of AD including acanthosis (epidermal thickening), hyperkeratosis (stratum corneum thickening), spongiosis (epidermal edema), and mixed dermal lymphocyte and eosinophil infiltration.

Methods described herein are generally performed on a subject in need thereof. A subject in need of the therapeutic methods described herein can be a subject having, diagnosed with, suspected of having, or at risk for developing an allergic disorder. A determination of the need for treatment will typically be assessed by a history and physical exam consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans. For example, the subject can be a human subject.

Generally, a safe and effective amount of an NK cell-stimulating agent is, for example, that amount that would cause the desired therapeutic effect in a subject while minimizing undesired side effects. In various embodiments, an effective amount of an NK cell-stimulating agent described herein can substantially inhibit symptoms of an allergic disorder, slow the progress of an allergic disorder, or limit the development of an allergic disorder.

According to the methods described herein, administration can be parenteral, pulmonary, oral, topical, intradermal, intramuscular, intraperitoneal, intravenous, subcutaneous, intranasal, epidural, ophthalmic, buccal, or rectal administration.

When used in the treatments described herein, a therapeutically effective amount of an NK cell-stimulating agent can be employed in pure form or, where such forms exist, in pharmaceutically acceptable salt form and with or without a pharmaceutically acceptable excipient. For example, the NK cell-stimulating agents of the present disclosure can be administered, at a reasonable benefit/risk ratio applicable to any medical treatment, in a sufficient amount to inhibit symptoms of an allergic disorder, slow the progress of an allergic disorder, limit the development of an allergic disorder, stimulate NK cells, boost NK cell function, increase an amount of NK cells, or improve symptoms of atopic dermatitis or other related allergic disorders.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4^{th} ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or a physician.

Administration of an IL-15 agonist can occur as a single event or over a time course of treatment. For example, an NK cell-stimulating agent can be administered daily, weekly, bi-weekly, or monthly. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

Treatment in accord with the methods described herein can be performed before, concurrent with, or after conventional treatment modalities for an allergic disorder.

An NK cell-stimulating agent can be administered simultaneously or sequentially with another agent, such as an antibiotic, an anti-inflammatory, an antibody (e.g., dupilumab), or another agent. For example, an NK cell-stimulating agent can be administered simultaneously with another agent, such as an antibiotic, an antibody (e.g., dupilumab), or an anti-inflammatory. Simultaneous administration can occur through administration of separate compositions, each containing one or more of an NK cell-stimulating agent, an antibiotic, an antibody (e.g., dupilumab), an anti-inflammatory, or another agent. Simultaneous administration can occur through administration of one composition containing two or more of an NK cell-stimulating agent, an antibiotic, an antibody (e.g., dupilumab), an anti-inflammatory, or another agent. An NK cell-stimulating agent can be administered sequentially with an antibiotic, an antibody (e.g., dupilumab), an anti-inflammatory, or another agent. For example, an NK cell-stimulating agent can be administered before or after administration of an antibiotic, an antibody (e.g., dupilumab), an anti-inflammatory, or another agent.

Administration

Agents and compositions described herein can be administered according to methods described herein in a variety of means known to the art. The agents and composition can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body.

As discussed above, administration can be parenteral, pulmonary, oral, topical, intradermal, intramuscular, intraperitoneal, intravenous, subcutaneous, intranasal, epidural, ophthalmic, buccal, or rectal administration.

Agents and compositions described herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the agent or composition in a manner similar to that used for delivering insulin or chemotherapy to specific organs or tumors. Typically, using such a system, an agent or composition can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

Agents can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006) Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to nontarget tissues; decrease irritation caused by the agent; decrease toxicity due to high initial doses of the agent; alter the immunogenicity of the agent; decrease dosage frequency; improve taste of the product; or improve shelf life of the product.

IL-15 superagonists (e.g., ALT-803) have been studied and are well tolerated in humans. As such, administration routes and doses can be as described therein (see e.g., Rommee et al. Blood Volume 131, Issue 23, 2018; Knudson et al. Expert Opin. On Biol. Ther. 2020 (7), incorporated herein by reference).

Screening

Also provided are methods for screening for NK cell-stimulating agents (e.g., IL-15 agonists) capable of stimulating NK cells, boosting NK cell function, increasing the amount of NK cells (e.g., in the subject, in the tissue or cells of a subject), or improving symptoms of atopic dermatitis or other related allergic disorders, such as allergic disorders that can be prevented or ameliorated with supraphysiologic levels of NK cells.

The subject methods find use in the screening of a variety of different candidate molecules (e.g., potentially therapeutic candidate molecules). Candidate substances for screening according to the methods described herein include, but are not limited to, known or unknown NK cell-stimulating agents, fractions of tissues or cells, nucleic acids, polypeptides, siRNAs, antisense molecules, aptamers, ribozymes, triple helix compounds, antibodies, and small (e.g., less than about 2000 MW, or less than about 1000 MW, or less than about 800 MW) organic molecules or inorganic molecules including but not limited to salts or metals.

Candidate molecules encompass numerous chemical classes, for example, organic molecules, such as small organic compounds having a molecular weight of more than 50 and less than about 2,500 Daltons. Candidate molecules can comprise functional groups necessary for structural interaction with proteins, particularly hydrogen bonding, and typically include at least an amine, carbonyl, hydroxyl, or carboxyl group, and usually at least two of the functional chemical groups. The candidate molecules can comprise cyclical carbon or heterocyclic structures and/or aromatic or polyaromatic structures substituted with one or more of the above functional groups.

A candidate molecule can be a compound in a library database of compounds. One of skill in the art will be generally familiar with, for example, numerous databases for commercially available compounds for screening (see e.g., ZINC database, UCSF, with 2.7 million compounds over 12 distinct subsets of molecules; Irwin and Shoichet (2005) J Chem Inf Model 45, 177-182). One of skill in the art will also be familiar with a variety of search engines to identify commercial sources or desirable compounds and classes of compounds for further testing (see e.g., ZINC database; eMolecules.com; and electronic libraries of commercial compounds provided by vendors, such as ChemBridge, Princeton BioMolecular, Ambinter SARL, Enamine, ASDI, Life Chemicals, etc.).

Candidate molecules for screening according to the methods described herein include both lead-like compounds and drug-like compounds. A lead-like compound is generally understood to have a relatively smaller scaffold-like structure (e.g., molecular weight of about 150 kD to about 350 kD) with relatively fewer features (e.g., less than about 3 hydrogen donors and/or less than about 6 hydrogen acceptors; hydrophobicity character x log P of about −2 to about 4) (see e.g., Angewante (1999) Chemie Int. ed. Engl. 24, 3943-3948). In contrast, a drug-like compound is generally understood to have a relatively larger scaffold (e.g., molecular weight of about 150 kD to about 500 kD) with relatively more numerous features (e.g., less than about 10 hydrogen acceptors and/or less than about 8 rotatable bonds; hydrophobicity character x log P of less than about 5) (see e.g., Lipinski (2000) J. Pharm. Tox. Methods 44, 235-249). Initial screening can be performed with lead-like compounds.

When designing a lead from spatial orientation data, it can be useful to understand that certain molecular structures are characterized as being “drug-like”. Such characterization can be based on a set of empirically recognized qualities derived by comparing similarities across the breadth of known drugs within the pharmacopeia. While it is not required for drugs to meet all, or even any, of these characterizations, it is far more likely for a drug candidate to meet with clinical success if it is drug-like.

Several of these “drug-like” characteristics have been summarized into the four rules of Lipinski (generally known as the “rules of fives” because of the prevalence of the number 5 among them). While these rules generally relate to oral absorption and are used to predict bioavailability of compound during lead optimization, they can serve as effective guidelines for constructing a lead molecule during rational drug design efforts such as may be accomplished by using the methods of the present disclosure.

The four “rules of five” state that a candidate drug-like compound should have at least three of the following characteristics: (i) a weight less than 500 Daltons; (ii) a log of P less than 5; (iii) no more than 5 hydrogen bond donors (expressed as the sum of OH and NH groups); and (iv) no more than 10 hydrogen bond acceptors (the sum of N and O atoms). Also, drug-like molecules typically have a span (breadth) of between about 8 Å to about 15 Å.

Compositions and methods described herein utilizing molecular biology protocols can be according to a variety of standard techniques known to the art (see e.g., Sambrook and Russel (2006) Condensed Protocols from Molecular Cloning: A Laboratory Manual, Cold Spring Harbor Laboratory Press, ISBN-10: 0879697717; Ausubel et al. (2002) Short Protocols in Molecular Biology, 5th ed., Current Protocols, ISBN-10: 0471250929; Sambrook and Russel (2001) Molecular Cloning: A Laboratory Manual, 3d ed., Cold Spring Harbor Laboratory Press, ISBN-10: 0879695773; Elhai, J. and Wolk, C. P. 1988. Methods in Enzymology 167, 747-754; Studier (2005) Protein Expr Purif. 41(1), 207-234; Gellissen, ed. (2005) Production of Recombinant Proteins: Novel Microbial and Eukaryotic Expression Systems, Wiley-VCH, ISBN-10: 3527310363; Baneyx (2004) Protein Expression Technologies, Taylor & Francis, ISBN-10: 0954523253).

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in or deleted from a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1: Natural Killer Cell Dysregulation is a Diagnostic and Treatment-Responsive Feature of Atopic Dermatitis

The following example describes the discovery that a reduction of blood natural killer (NK) cells are a diagnostic feature of AD that recovers in patients following type 2 cytokine blockade with dupilumab. It was also discovered that IL-15 superagonists stimulate NK cells, resulting in an increase of NK cells and function. It was shown that treatment with the IL-15 superagonist, IL-15:sIL-15Rα complex, attenuates AD-like disease

Multidimensional CyTOF and RNA sequencing of NK cells revealed selective shifts in subpopulations and transcriptional changes indicative of activation-induced cell death (AICD) in AD. Further, NK cell deficiency in a murine AD model resulted in an expansion of group 2 innate lymphoid cells (ILC2s) in the skin, suggesting that NK cells may provide an important immunoregulatory function. Collectively, this study provides new insight into the relationship between type 2 inflammation and NK cells that has potential diagnostic applications and use in clinical trials to track treatment response.

Nurturing NK Cells to Treat Atopic Dermatitis

The skin condition atopic dermatitis (AD) is driven by a type 2 immune response. Described herein is high-dimensional immune profiling of patients with AD and revealed deficiencies in certain subsets of natural killer (NK) cells. NK cells showed signs of activation-induced cell death and were restored in patients that responded to immunotherapy. Also described herein is the discovery that circulating NK cells were also decreased in a mouse AD model and boosting NK cells with an IL-15 superagonist ameliorated symptoms in the mice. These results suggest that strategies to restore NK cells could help rebalance immunity in AD.

Abstract

Atopic dermatitis (AD) is a widespread, chronic skin disease associated with aberrant allergic inflammation. Current treatments involve either broad or targeted immunosuppression strategies. However, enhancing the immune system to control disease remains untested. Here, it was demonstrated that patients with AD harbor a blood natural killer (NK) cell deficiency that both has diagnostic value and improves with therapy. Multidimensional protein and RNA profiling revealed subset-level changes associated with enhanced NK cell death. Murine NK cell deficiency was associated with enhanced type 2 inflammation in the skin, suggesting that NK cells play a critical immunoregulatory role in this context. On the basis of these findings, an NK cell-boosting interleukin-15 (IL-15) superagonist was used and marked improvement in AD-like disease in mice was observed. These findings reveal a previously unrecognized application of IL-15 superagonism, currently in development for cancer immunotherapy, as an immunotherapeutic strategy for AD.

Introduction

Atopic dermatitis (AD) or eczema is the most common inflammatory skin disorder, has a substantial negative impact on patients' quality of life, and costs $5.3 billion annually in the United States (1-3). AD is characterized by elevated production of the type 2 cytokines interleukin-4 (IL-4), IL-5, and IL-13, which promote AD pathogenesis (4). Although classically associated with adaptive T helper type 2 cell responses, more recent work has identified that innate immune cell populations such as basophils and group 2 innate lymphoid cells (ILC2s) are major sources of these cytokines in AD (5-7). As a result, current treatment strategies in AD have focused exclusively on either broad or selective immunosuppression to combat pathologic type 2 inflammation. However, although it is well understood that type 2 immune cells promote AD pathogenesis, the endogenous mechanisms that maintain immune homeostasis and restrain inflammation in AD remain poorly defined. Beyond skin lesions, moderate-to-severe AD is associated with a systemic immune response involving increased blood eosinophils, elevated immunoglobulin E (IgE), and the development of other atopic disorders such as asthma and food allergy (8-10). The identification of immune pathways that suppress type 2 inflammation in AD may reveal previously unrecognized cellular pathways that could be therapeutically targeted to ameliorate AD and potentially other allergic diseases.

In addition to robust type 2 inflammation, patients with AD are known to exhibit diminished antiviral immunity and heightened risk for the development of severe disseminated herpesvirus (eczema herpeticum) and vaccinia virus (eczema vaccinatum) infections (11, 12). Natural killer (NK) cells are innate lymphocytes that comprise 5 to 10% of the circulating peripheral blood mononuclear cells (PBMCs) and critically promote antiviral immunity, in part through the production of interferon-γ (IFN-γ). Alterations in NK cell numbers, killing capacity, and IFN-γ production have been described in patients with AD since the early 1980s (13-16); however, the specificity and clinical applications of this feature have been largely ignored. IFN-γ has been shown to suppress type 2 inflammation (17-19), suggesting that NK cell dysregulation is a functionally relevant feature of AD. Recent studies have demonstrated that NK cells and IFN-γ can restrain ILC2 responses in vitro and during allergic lung inflammation (17, 18, 20), implicating NK cells broadly in regulating allergic inflammation. It was hypothesized that NK cells are dysfunctional in patients with AD and contribute to the disease process.

Results

NK Cell Deficiency is a Diagnostic Feature of Moderate-to-Severe AD

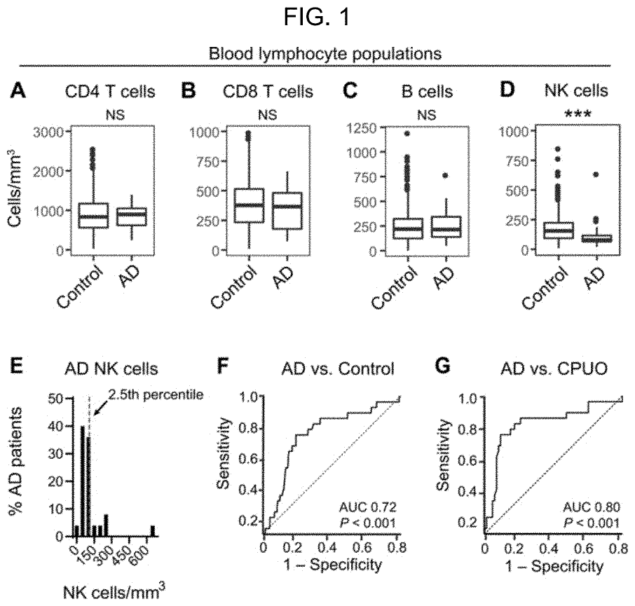

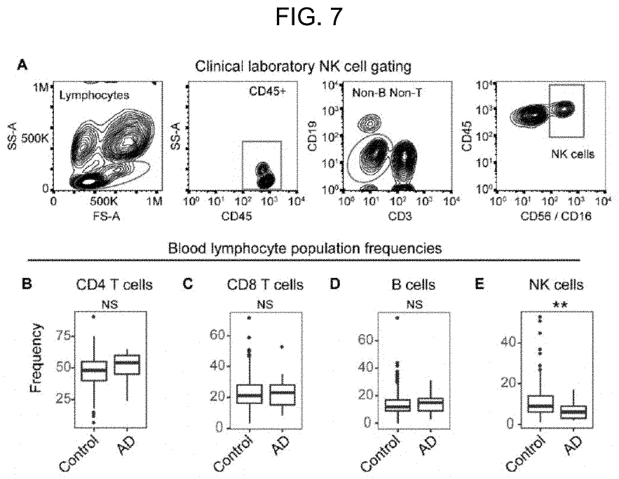

First, a comprehensive analysis of blood lymphocyte subpopulations was performed in 25 adult patients with moderate-to-severe AD (43±3.4 years; 52% female) and compared them to a control cohort of 363 subjects without AD (50±1 years; 64% female) seen during the study period (see e.g., FIG. 1A-FIG. 1D, FIG. 7). This analysis revealed that patients with AD exhibited a marked and selective reduction in the number of blood NK cells (average 116.7±24.6 NK cells/mm³) compared to controls (178.1±0.7 NK cells/mm³; P=0.00016) (see e.g., FIG. 1D, FIG. 7E), and 72% of the AD cohort had NK cell numbers below the 2.5th percentile of normal (<105 NK cells/mm³) (see e.g., FIG. 1E). An analysis of diagnostic value using a receiver operating characteristic (ROC) curve indicated that low blood NK cell numbers can effectively differentiate patients with AD from an all-comers cohort of patients without AD seen across multiple hospital departments (see e.g., FIG. 1F). Low blood NK cells were even more effective at distinguishing AD from those with moderate-to-severe chronic pruritus of unknown origin (CPUO), another pruritic disorder that currently lacks distinguishing biomarkers (see e.g., FIG. 1G) (21).

Patients with AD Exhibit Alterations in Specific Subpopulations of NK Cells

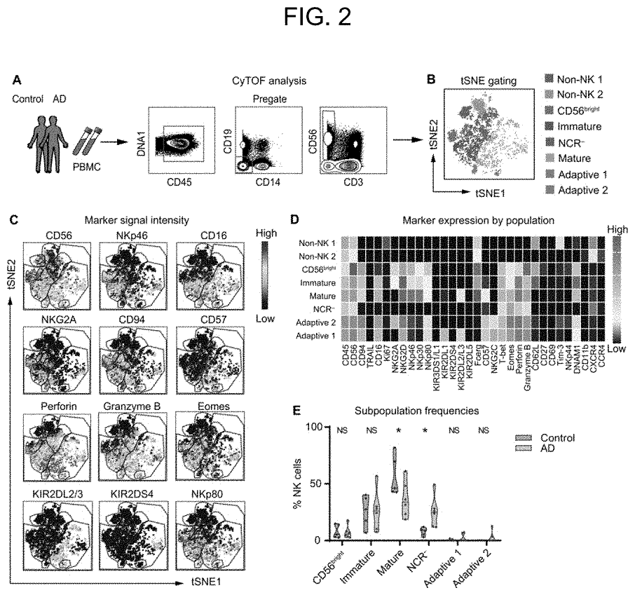

Two functionally distinct NK cell subpopulations, CD56^{bright} and CD56^{dim} NK cells, specialize in cytokine production and target cell killing, respectively (22). Beyond this initial binary classification, recent studies using high-dimensional mass cytometry (CyTOF) technology have revealed a large degree of phenotypic heterogeneity in human NK cells (23). This variation is, in part, due to individual genetic variation, such as human leukocyte antigen (HLA) and killer cell Ig-like receptor (KIR) haplotypes, and environmental exposures (e.g., cytomegalovirus) that shape the NK cell surface receptor repertoire (24). Therefore, CyTOF was used on peripheral blood from patients with AD and controls (see e.g., FIG. 2A, TABLE 1) in conjunction with viSNE for dimensionality reduction to characterize NK cell subpopulations in AD. NK cells were enriched from total PBMCs by preselecting for lineage (Lin)⁻ CD56⁺ cells before viSNE analysis (see e.g., FIG. 2A, FIG. 2B).

TABLE 1
Demographics for subjects analyzed in FIG. 2, NK cell CyTOF.

| Characteristic | Control | AD | |
|---|---|---|---|
| Subjects (N) | 5 | 6 | |
| Mean age (yr) | 47.8 (5.6) | 52.8 | (4.9) |
| Percent female | 20% | 50% | |
| EASI score | NA | 44.7 | (2.6) |
| NRS Itch score | NA | 8.4 | (0.8) |
| IgE (IU/mL) | NA | 8,976.2 | (4,861.4) |

*Data are mean (SEM), NA = Not applicable, NRS = Numerical Rating Scale in which 0 = no itch and 10 = worst imaginable itch, EASI = Eczema Area and Severity Index

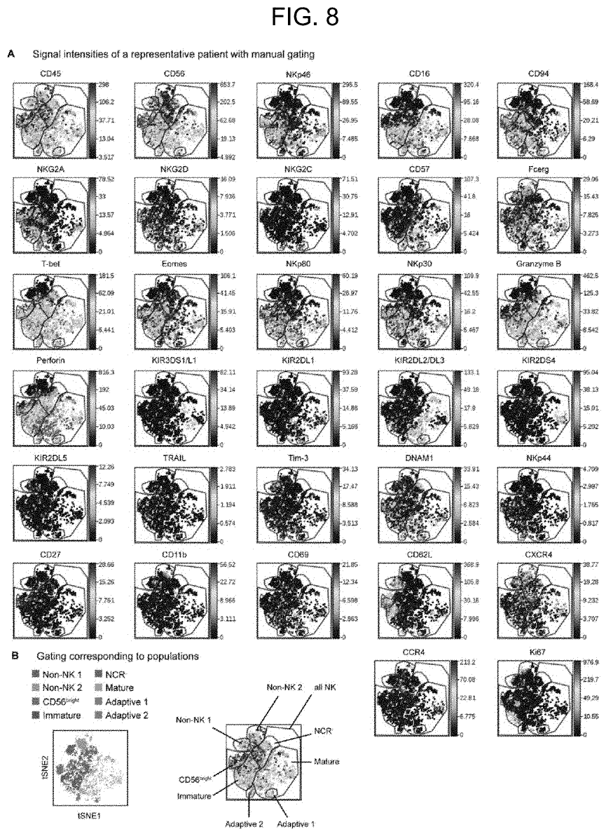

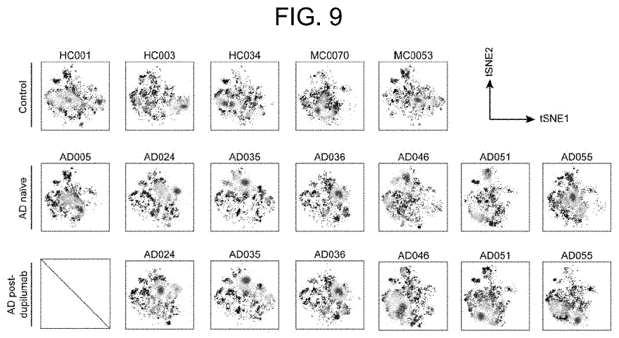

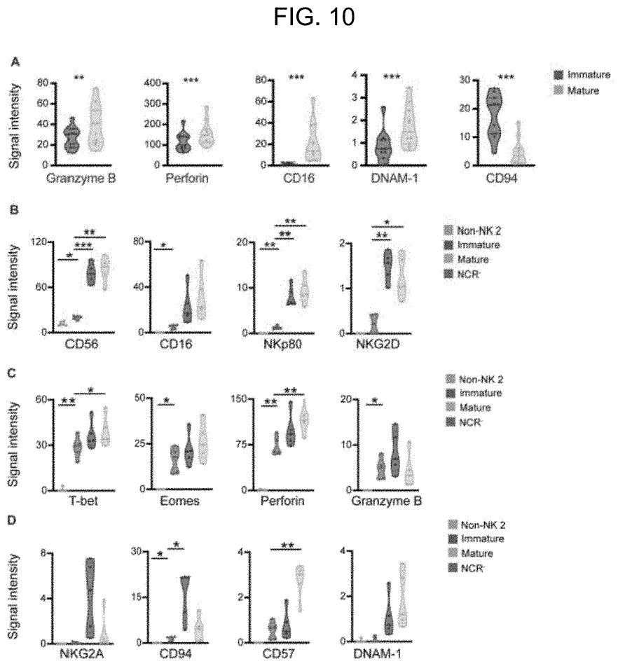

Manual gating was performed on the basis of previously described subsets of CD56^{bright} immature CD56^{dim} mature CD56^{dim}, and adaptive NK cells (see e.g., FIG. 2C, FIG. 8, FIG. 9) (25). Briefly, mature CD56^{dim} cells are characterized by enhanced lytic granule protein contents, including granzyme B and perforin, expression of CD57, and expression of KIRs (see e.g., FIG. 2D). To identify comparable populations between genetically diverse donors, it was considered that cells expressing any of the KIRs in the CyTOF panel to be mature. In contrast, immature CD56^{dim} cells have enhanced NKG2A/CD94 expression in the absence of CD57 and KIRs (see e.g., FIG. 10A) (26-28). Last, adaptive NK cells, which are expanded in response to cytomegalovirus exposure, are classically defined by NKG2C expression in the absence of FcεRy (29-32).

The viSNE analysis and gating approach identified a total of eight distinct cellular subsets (see e.g., FIG. 2D). Two of these were determined to be non-NK cell populations, because they were uniformly negative for all canonical NK cell markers other than CD56 (see e.g., FIG. 2D). These non-NK populations are likely composed of CD56⁻ populations captured through a generous pregate, although they could also contain CD56-expressing innate lymphocytes present at very low frequencies in the blood (33-35). Of the six NK cell populations, patients with AD exhibited a selective reduction in mature CD56^{dim} NK cells, but not CD56^{bright} immature CD56^{dim}, or adaptive populations (see e.g., FIG. 2E). Patients with AD also displayed elevated frequencies of a nonclassical natural cytotoxicity receptor-negative population, which was termed NCR⁻, that lacked expression of natural cytotoxicity receptors (NCRs) (NKp30, NKp80, and NKp46), NKG2D, and CD16 but did express NK cell-associated transcription factors (T-bet and Eomes) and effector molecules (granzyme B and perforin) (see e.g., FIG. 2E, FIG. 10B-FIG. 10D). Together, these findings demonstrate that patients with AD have not only a global reduction in their blood NK cells but also a shift in subpopulations of NK cells that reflects a loss of mature CD56^{dim} effector cells.

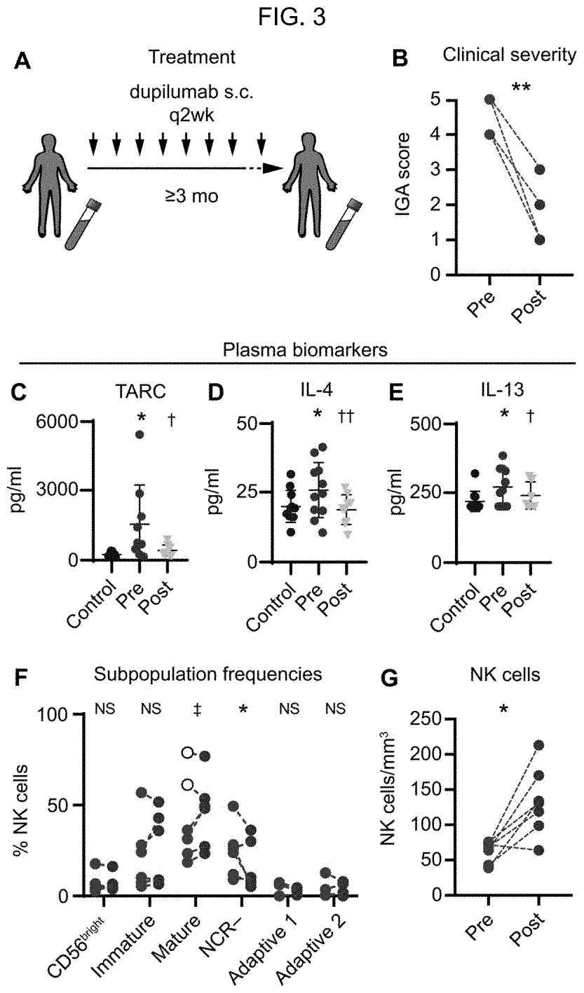

Type 2 cytokine blockade reverses NK cell defects in patients with AD Dupilumab is an anti-IL-4 receptor α (IL-4Rα) monoclonal antibody (mAb) that is highly effective for the treatment of moderate-to-severe AD (36-38). It was therefore sought to investigate whether the NK cell alterations observed in patients' blood are reversed by dupilumab treatment. Patients with AD were examined before and after receiving dupilumab (see e.g., FIG. 3, TABLE 2) for disease severity, as measured by the Investigator Global Assessment (IGA) score, clinical flow cytometry, and CyTOF. After treatment, all patients exhibited improvement in their IGA score (see e.g., FIG. 3B) as well as a reduction in the inflammatory, AD-associated serum biomarkers TARC (CCL17), IL-4, and IL-13 (see e.g., FIG. 3C-FIG. 3E, TABLE 3). In association with the clinical response, the majority (67%) of patients demonstrated recovery of mature CD56^{dim} NK cells, whereas all patients (100%) had a significant reduction in the frequency of the NCR⁻ population by CyTOF analysis (P=0.0279; FIG. 3F). The two patients whose mature NK cells did not recover after dupilumab had the highest numbers of total NK cells and highest frequency of the mature subset before treatment. Although the recovery of mature CD56^{dim} NK cells was not statistically significant for the whole cohort (P=0.4498), there was a significant treatment effect on mature CD56^{dim} NK cells in patients with NK cells below the ROC diagnostic cutoff of 95 cells/mm³ (P=0.0391), suggesting that the degree of NK cell deficiency may affect this population's response to treatment. Notwithstanding this, there was a significant recovery of the total NK cell population as determined by clinical flow cytometry after dupilumab treatment (P=0.0156) (see e.g., FIG. 3G). This demonstrates that NK cell deficiency associated with AD is reversible by type 2 cytokine blockade.

TABLE 2
Demographics for subjects analyzed in FIG. 3,
NK cell CyTOF before/after dupilumab treatment.

| Characteristic | AD | |
|---|---|---|
| Subjects (N) | 6 | |
| Mean age (yr) | 45.3 | (6.0) |
| Percent female | 50% | |
| EASI score | 39.7 | (4.7) |
| Pre-treatment NRS Itch | 8.0 | (0.8) |
| score | | |
| IgE (IU/mL) | 8,316.1 | (4,990.7) |

*Data are mean (SEM), NRS = Numerical Rating Scale in which 0 = no itch and 10 = worst imaginable itch, EASI = Eczema Area and Severity Index

TABLE 3
Demographics for subjects analyzed in FIG. 3, plasma cytokines.

| Characteristic | Control | AD | |
|---|---|---|---|
| Subjects (N) | 11 | 10 | |
| Mean age (yr) | 37.1 (4.0) | 43.7 | (4.7) |
| Percent female | 54.5 | 45.5% | |
| EASI score | NA | 31.4 | (5.7) |
| Pre-treatment NRS Itch | NA | NA | |
| score | | | |
| IgE (IU/mL) | NA | 4,566.5 | (2,604) |

*Data are mean (SEM), NA = Not available, NRS = Numerical Rating Scale in which 0 = no itch and 10 = worst imaginable itch, EASI = Eczema Area and Severity Index

AD NK Cells Exhibit Cellular Features of Activation-Induced Cell Death

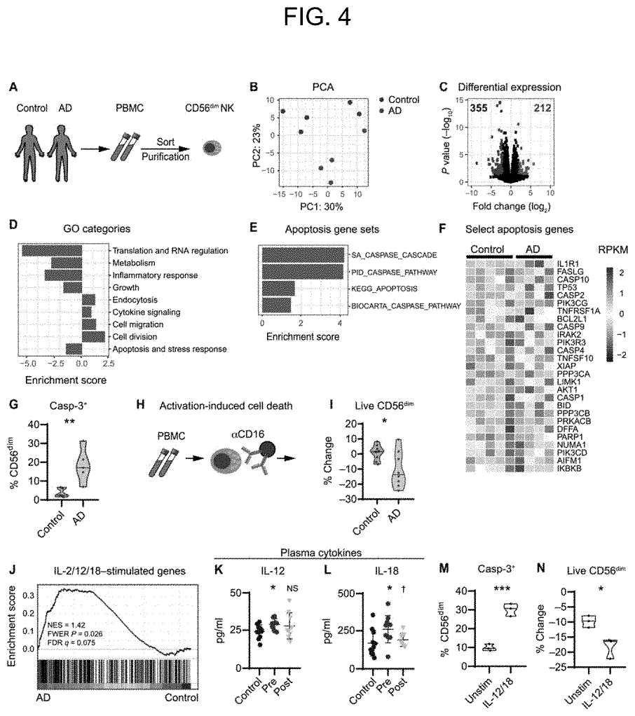

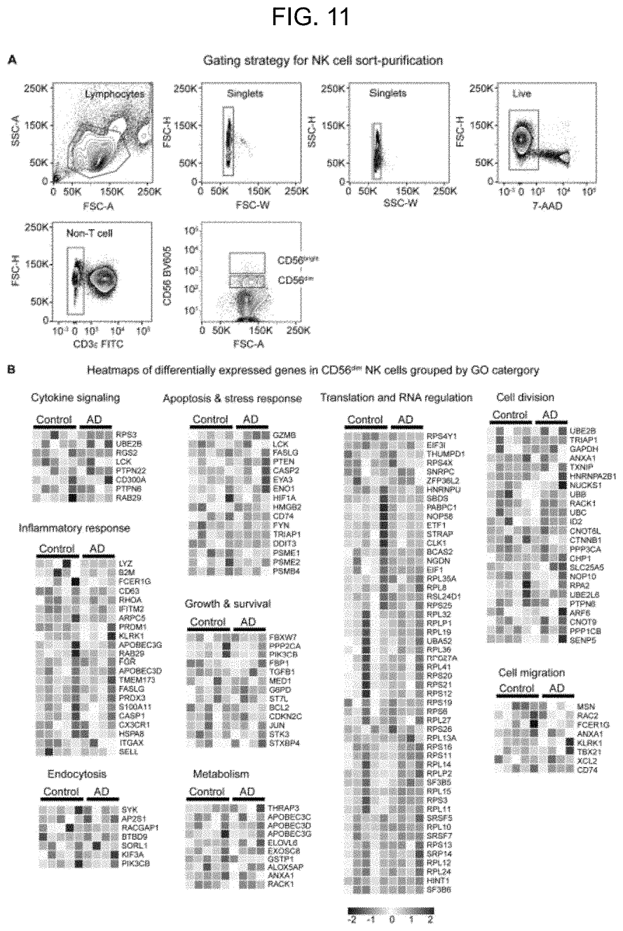

To identify cellular programs that may underlie the loss of mature CD56^{dim} NK cells in AD, RNA sequencing (RNA-seq) of sort-purified CD56^{dim} NK cells from both patients with AD and control individuals was performed (see e.g., FIG. 4A, FIG. 11A, TABLE 4). Principal components analysis (PCA) revealed distinct transcriptional profiles between AD and control NK cells (see e.g., FIG. 4B) with a total of 567 differentially expressed genes (P<0.05) (see e.g., FIG. 4C). Genes differentially expressed between AD-associated and control NK cells were enriched for gene ontology (GO) terms associated with endocytosis, cytokine signaling, cell migration, and cell division, suggestive of an enhanced activation state (see e.g., FIG. 4D, FIG. 11B). Together with the selective reduction in mature NK cells observed by CyTOF, it was hypothesized that NK cells from patients with AD may be undergoing activation-induced cell death (AICD).

TABLE 4
Demographics for subjects analyzed in FIG. 4, NK cell RNA-seq.

| Characteristic | Control | AD | |
|---|---|---|---|
| Subjects (N) | 5 | 4 | |
| Mean age (yr) | 56.4 (4.0) | 32.8 | (4.8) |
| Percent female | 60% | 80% | |
| EASI score | NA | 30.2 | (4.3) |
| Pre-treatment NRS Itch | NA | 8.0 | (0.6) |
| score | | | |
| IgE (IU/mL) | NA | 6,381.5 | (3,318.3) |

*Data are mean (SEM), NA = Not available, NRS = Numerical Rating Scale in which 0 = no itch and 10 = worst imaginable itch, EASI = Eczema Area and Severity Index

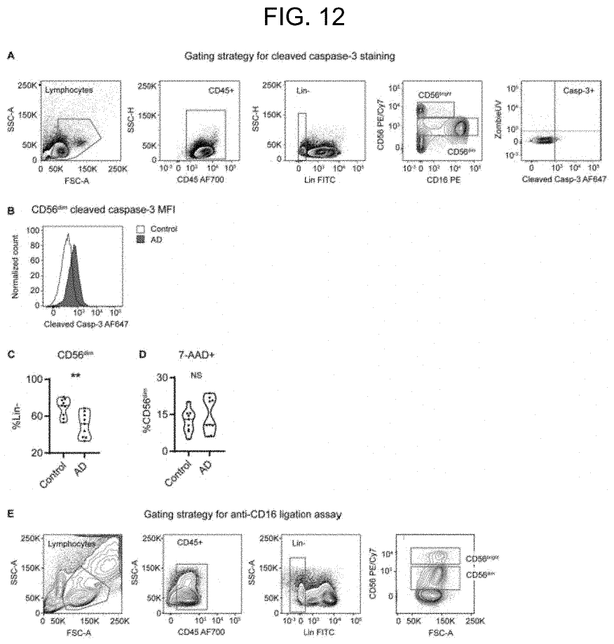

In support of this, gene set enrichment analyses (GSEAs) revealed that caspase-associated and apoptotic gene sets were enriched in AD CD56^{dim} NK cells (see e.g., FIG. 4E). Both initiator and effector caspases increased in expression along with stress-induced genes such as TP53, BCL2L1, and PARP1 (see e.g., FIG. 4F). To confirm these results, flow cytometry was performed on blood from AD and healthy control donors and increased activity of the proapoptotic effector caspase-3 in AD CD56^{dim} NK cells was found (see e.g., FIG. 4G, FIG. 12A, FIG. 4B, TABLE 5). However, despite the reduced frequency of total CD56^{dim} cells in their PBMCs (see e.g., FIG. 12C), an increase in dead CD56^{dim} NK cells by 7-aminoactinomycin D (7-AAD) staining at steady-state from AD donors was not observed (see e.g., FIG. 12D). Therefore, it was hypothesized that NK cell death in AD may require additional stimuli. NK cells have been reported to undergo AICD in response to ligation of the low-affinity Fc receptor CD16, a key mediator of antibody-dependent cellular cytotoxicity (39). A selective reduction in live CD56^{dim} NK cells from AD blood was observed compared to healthy control blood in response to CD16 ligation (see e.g., FIG. 4H, FIG. 4I, FIG. 12E, TABLE 6). These studies indicate that AD-associated NK cells exhibit a baseline proapoptotic phenotype and are more susceptible to AICD.

TABLE 5
Demographics for subjects analyzed in
FIG. 4, cleaved caspase-3 staining.

| Characteristic | Control | AD | |
|---|---|---|---|
| Subjects (N) | 5 | 5 | |
| Mean age (yr) | 27.6 (1.9) | 34.0 | (8.9) |
| Percent female | 60% | 80% | |
| EASI score | NA | 3.2 | (0.4) |
| Pre-treatment NRS Itch | NA | 9.0 | (0.9) |
| score | | | |
| IgE (IU/mL) | NA | 6,733.9 | (4,354.6) |

*Data are mean (SEM), NA = Not applicable, NRS = Numerical Rating Scale in which 0 = no itch and 10 = worst imaginable itch, IGA = Investigator's Global Assessment score using a 5-point scale in which 0 = no disease and 4 = severe disease.

TABLE 6
Demographics for subjects analyzed in FIG. 4, CD16 ligation.

| Characteristic | Control | AD | |
|---|---|---|---|
| Subjects (N) | 9 | 8 | |
| Mean age (yr) | 65.5 (2.2) | 49.3 | (4.6) |
| Percent female | 33.3% | 57.1% | |
| EASI score | NA | 36.9 | (6.2) |
| Pre-treatment NRS Itch | NA | 7.7 | (0.8) |
| score | | | |
| IgE (IU/mL) | NA | 11,118.7 | (4,770.0) |

*Data are mean (SEM), NA = Not applicable, NRS = Numerical Rating Scale in which 0 = no itch and 10 = worst imaginable itch, EASI = Eczema Area and Severity Index

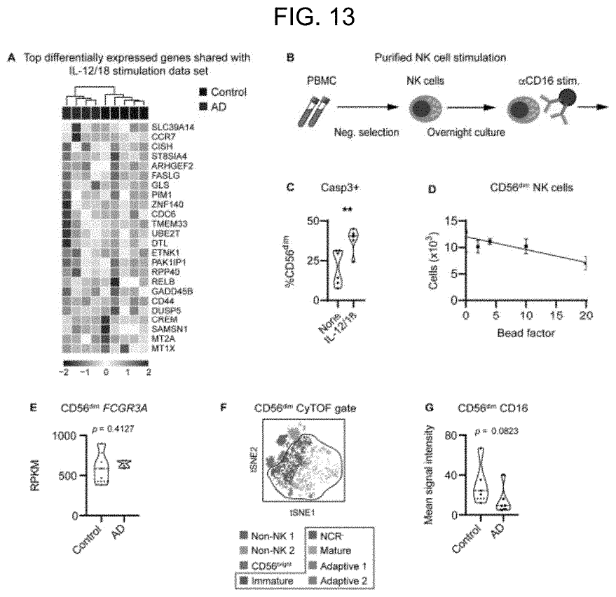

Previous studies have demonstrated that AICD of NK cells requires priming by activating cytokines such as IL-2 and IL-12 (39). RNA-seq data from control and AD CD56^{dim} NK cells to a previously published dataset derived from human NK cells that were stimulated in vitro with IL-2, IL-12, and IL-18 were compared (40). Using GSEA, significant enrichment of this cytokine-stimulated gene set in the AD NK cell transcriptomes were found (P=0.026) (see e.g., FIG. 4J, FIG. 13A). It was also observed that there was elevated IL-12 (see e.g., FIG. 4K) and IL-18 (see e.g., FIG. 4L) in the sera of patients with AD compared to sera from healthy controls (TABLE 3). Furthermore, IL-18 in patients with AD decreased after type 2 cytokine blockade (see e.g., FIG. 4L), suggesting that IL-18 may be associated with disease activity. To test whether elevated IL-12 and IL-18 may contribute to patients' NK cell pathology, control NK cells were preactivated with IL-12 and IL-18 and increased cleaved caspase-3 staining was observed (see e.g., FIG. 4M) and loss of NK cells after CD16 ligation (see e.g., FIG. 4N) in both bulk PBMCs and purified NK cell cultures (see e.g., FIG. 13B-FIG. 13D). In addition, although they had a similar amount of FCGR3A mRNA (see e.g., FIG. 13E), CD56^{dim} NK cells from patients with AD had, on average, less CD16 surface protein (see e.g., FIG. 13F, FIG. 13G). Because CD16 is known to be posttranslationally regulated by proteolytic cleavage in response to activation (41), this is consistent with an activated state of CD56^{dim} NK cells in vivo. Together, these findings suggest that, in the context of AD, blood CD56^{dim} NK cells are exposed to elevated IL-12 and IL-18 and primed for AICD.

NK Cells are Enriched in Lesional AD Skin and Limit Type 2 Inflammation

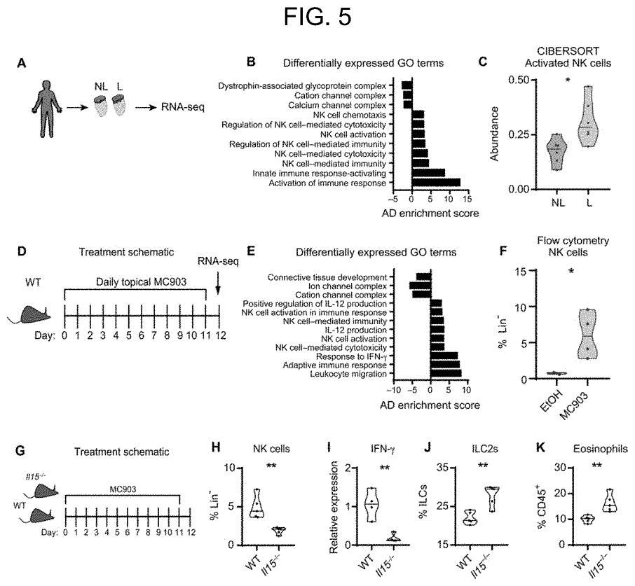

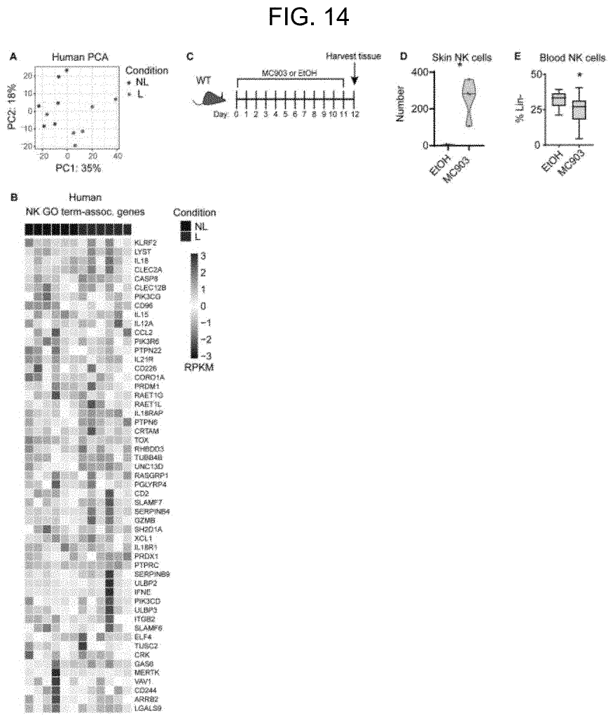

The strong association between AD and systemic NK cell dysregulation prompted us to investigate whether NK cells are altered in AD skin lesions. To examine this, RNA-seq of paired lesional and nonlesional skin biopsies from six patients with AD was performed to look for transcriptional evidence of NK cell activity (see e.g., FIG. 5A, TABLE 7). PCA readily distinguished the transcriptome of lesional skin from nonlesional skin (see e.g., FIG. 14A). To determine which pathways are altered in lesional skin, differentially enriched GO terms were evaluated. Among the most highly enriched GO terms, a number involved NK cell activation, cytotoxicity, and migration (see e.g., FIG. 5B). Analysis of the differentially expressed genes driving this GO term enrichment revealed genes known to be involved in NK cell function, such as CD226(DNAM-1), CRTAM, PRDM1, and GZMB (see e.g., FIG. 14B). To test whether these gene expression changes may be indicative of enhanced NK cell abundance in the lesional skin, the CIBERSORT platform was used to generate a predicted cellular composition of the complex tissue (42). Consistent with the GO enrichment analysis, CIBERSORT imputed an enrichment of activated NK cells in lesional AD skin compared to nonlesional skin (see e.g., FIG. 5C).

TABLE 7
Demographics for subjects analyzed in FIG. 5, skin RNA-seq.

| Characteristic | AD | |
|---|---|---|
| Subjects (N) | 6 | |
| Mean age (yr) | 44.8 | (8.4) |
| Percent female | 50% | |
| EASI score | 34.6 | (4.8) |
| Pre-treatment NRS Itch | NA | |
| score | | |
| IgE (IU/mL) | 4,192.2 | (3,221.5) |

*Data are mean (SEM), NA = Not available, NRS = Numerical Rating Sc 0 = no itch and 10 = worst imaginable itch, EASI = Eczema Area and Severity Index

This approach was validated by repeating it on a well-established murine model of AD-like disease. AD was induced using a standardized protocol of topical MC903 (calcipotriol) application daily for 12 days (see e.g., FIG. 5D, FIG. 14C) (43, 44). This model recapitulates the central features of AD including erythema (redness), scaling, blood eosinophilia, serum IgE elevation, itch behavior (pruritus), and histopathologic features of AD including acanthosis (epidermal thickening), hyperkeratosis (stratum corneum thickening), spongiosis (epidermal edema), and mixed dermal lymphocyte and eosinophil infiltration (5, 6, 43, 45). Similar to human AD skin, GO terms related to NK cell function were enriched in murine AD-like skin such as “NK cell activation” and “NK cell activation in immune response” (see e.g., FIG. 5E). Last, as predicted by the RNA-seq signature, AD-like skin exhibited a higher frequency (see e.g., FIG. 5F) and number (see e.g., FIG. 14D) of NK1.1⁺ NK cells compared to skin of control mice by traditional flow cytometry. Together, these findings suggest that NK cells are enriched and activated in lesional AD and AD-like skin of both humans and mice, respectively.

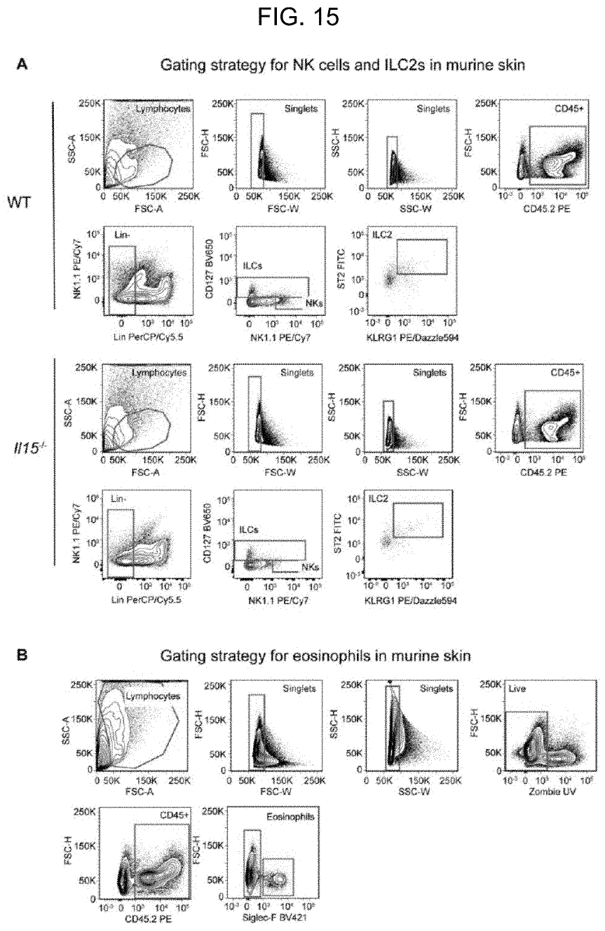

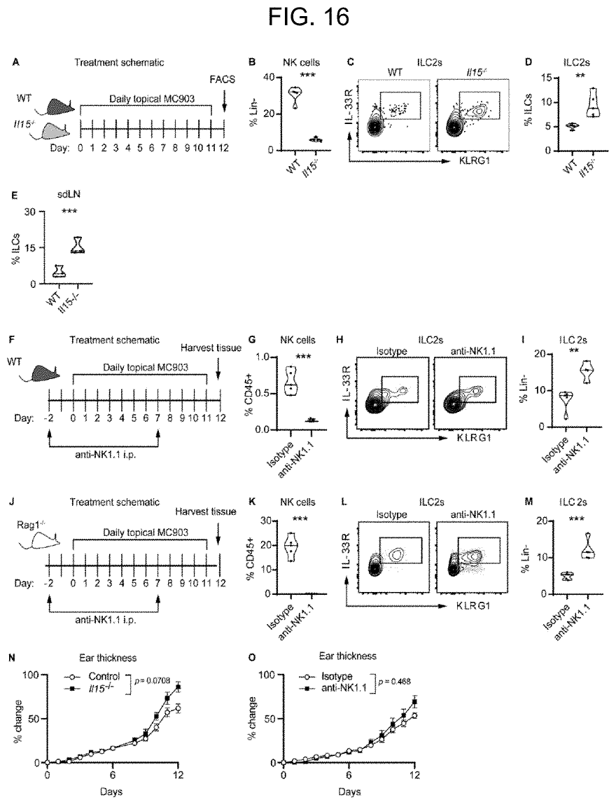

It was next asked whether the systemic loss of NK cells that was observed in patients with AD affects the disease process. Recent studies have found that NK cells and IFN-γ suppress ILC2 proliferation and cytokine production in vitro and in allergic lung inflammation (17-19). Because ILC2s are critical pathogenic drivers of AD (5-7), it was hypothesized that systemic NK cell deficiency in patients with AD may contribute to their inflammatory skin disease. First, peripheral blood NK cells were measured in the murine model and found that, like humans, mice given MC903 had a decreased frequency of circulating NK cells compared to controls overtime (see e.g., FIG. 14E). To determine whether this reduction contributes to disease, AD-like disease was induced in Il15^{-/-} mice, which are developmentally NK cell-deficient (see e.g., FIG. 5G) (46). As expected, these mice had substantial reductions in both NK cells (see e.g., FIG. 5H, FIG. 15A) and Ifng expression (see e.g., FIG. 5I) in lesional skin. In addition, an increase in ILC2s was observed (see e.g., FIG. 5J, FIG. 15A) and eosinophils (see e.g., FIG. 5K, FIG. 15B) in skin lesions as well as the skin-draining lymph nodes (see e.g., FIG. 16A-FIG. 16D) of Il15^{-/-} mice compared to controls. Il15^{-/-} mice also had an increased frequency of ILC2s at steady state (see e.g., FIG. 16E), suggesting that NK cell regulation of ILC2s may be a homeostatic function. Furthermore, to confirm the effect of NK cell deficiency on ILC2s during AD-like disease, NK cells were depleted from both wild-type (WT) and T cell-deficient Rag1^{-/-} mice during induction of AD-like disease using anti-NK1.1 mAbs and observed similar effects of NK cell reduction on ILC2 frequencies (see e.g., FIG. 16F-FIG. 16M). Together, these findings demonstrate that NK cells limit innate type 2 inflammation in the skin in a murine model of AD.

IL-15 Superagonism Promotes NK Cell-Dependent Resolution of AD-Like Inflammation

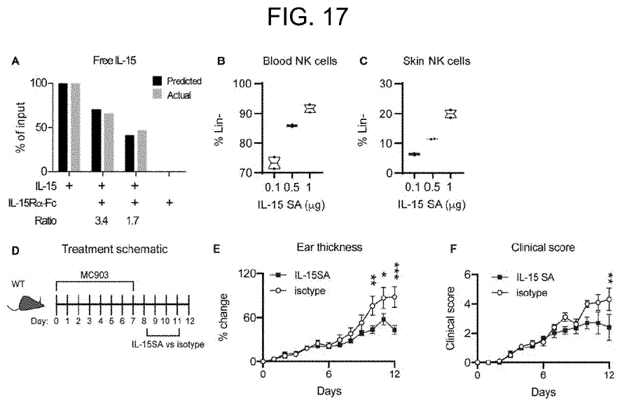

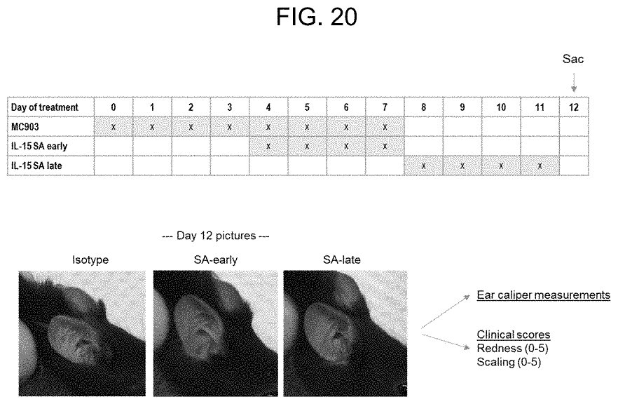

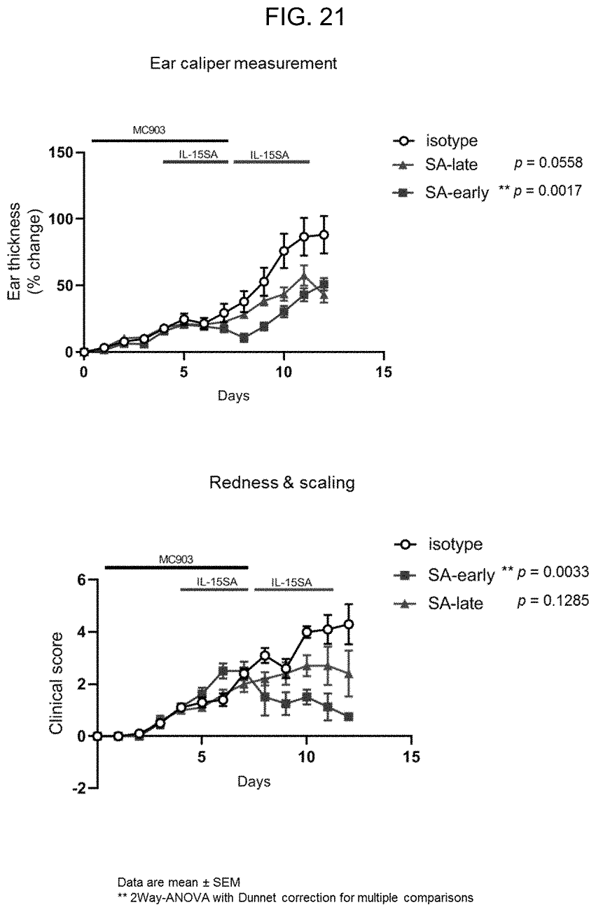

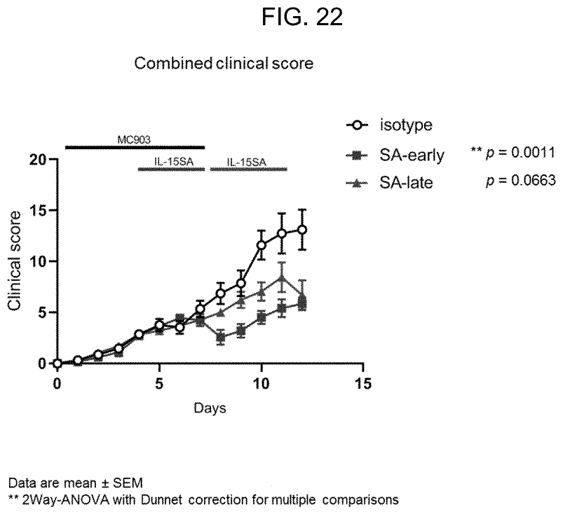

Despite the robust effect of NK cell depletion on skin ILC2s and eosinophils, a notable exacerbation of clinical disease in NK cell-deficient mice was not observed (see e.g., FIG. 16N, FIG. 16O). This suggests that physiologic NK cell responses are insufficient to control disease under AD-like conditions. Therefore, it is presently believed that boosting NK cells to supraphysiologic levels can promote their regulatory function and ameliorate disease. IL-15 superagonists (SAs) are an established method to promote NK cell survival and proliferation in vivo (47, 48) and are currently being used in clinical trials either alone or in combination with checkpoint inhibitors for cancer immunotherapy (49-51). However, whether an NK cell immunotherapy strategy could be applied to allergic diseases such as AD remains unexplored. Thus, a murine IL-15 SA was generated, in which a soluble IL-15Rα-Fc complex is stably loaded with IL-15 (see e.g., FIG. 6A, FIG. 17A), which was confirmed to be able to induce NK cell expansion in vivo in a dose-dependent manner (see e.g., FIG. 17B, FIG. 17C). After 4 days of AD-like disease induction in WT mice, systemic murine IL-15 SA or an IgG1 isotype control was administered and disease progression was monitored (see e.g., FIG. 6B). Mice that received IL-15 SA had more NK cells in the blood (see e.g., FIG. 6C) and skin (see e.g., FIG. 6D). They also had reduced numbers of skin ILC2s and eosinophils (see e.g., FIG. 6E, FIG. 6F) compared to mice that received isotype control treatment. These cellular changes were accompanied by a robust reduction in disease severity (see e.g., FIG. 6G), including clinical scoring (see e.g., FIG. 6H, FIG. 21), skin thickness (see e.g., FIG. 6I, FIG. 21), and histopathology (see e.g., FIG. 6J, FIG. 6K). Initiation of IL-15 SA treatment at an even later time point, on experimental day 8, was also able to reduce disease symptoms (see e.g., FIG. 17D-FIG. 17F, FIG. 20, FIG. 21), indicating that this method has robust therapeutic potential in the setting of established disease. As such, the IL-15 superagonist (IL-15:sIL-15Ra, see e.g., FIG. 19A) attenuates AD-like disease in wild type mice (see e.g., FIG. 20, FIG. 21, FIG. 22). Here is also described, together, early administration (day 4) and later administration (day 8) (see e.g., FIG. 20-FIG. 22).

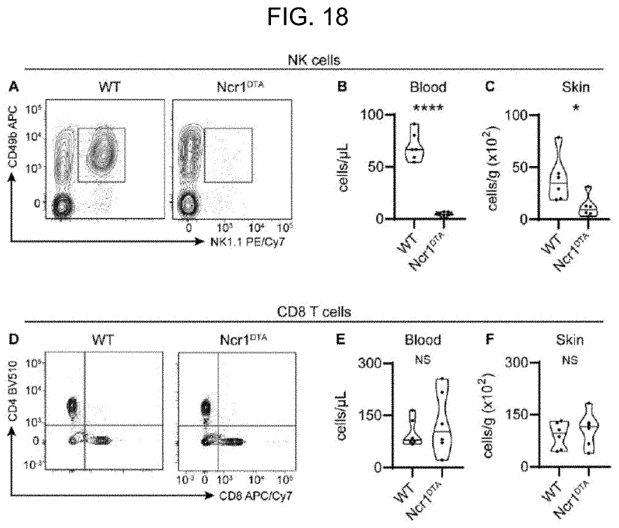

Although IL-15 SA treatments have been shown to primarily target NK cells in both mice and patients (47, 51), IL-15 is also important in generating memory CD8 T cell responses (46). To determine the relative contributions of NK cells and CD8 T cells in IL-15 SA-mediated disease reduction, IL-15 SA was administered to Cd8^{-/-} mice after AD-like disease induction (see e.g., FIG. 6L). Despite the lack of CD8 T cells, IL-15 SA significantly reduced ear thickness (P=0.0189; FIG. 6M) and clinical scores (P=0.0138; FIG. 6N), suggesting that NK cells are sufficient to mediate the therapeutic effect. To test whether NK cells are specifically required, NK cell-deficient mice were generated using Ncr1-iCre mice crossed with Rosa-stop-floxed-DTA mice (Ncr1^{DTA}) (see e.g., FIG. 6O, FIG. 18). Conditional deletion of NK cells abrogated the ability of IL-15 SA to reduce both ear thickness (see e.g., FIG. 6P) and clinical scores (see e.g., FIG. 6Q). Collectively, these studies indicate that restoring NK cell deficiency through IL-15 superagonism is an effective and promising therapeutic strategy for AD.

Discussion

AD is a systemic immune disorder in a family of type 2 inflammatory conditions including asthma and food allergy that are characterized by elevated IgE, eosinophilia, and a predisposition for allergen sensitization across barrier surfaces. Although most of the research on AD pathology thus far has focused on cutaneous mechanisms of barrier dysfunction and inflammatory cell recruitment, how the systemic immune system in AD is negatively affected is poorly defined. In this study, it is shown that low peripheral blood NK cells in patients with AD have diagnostic value in distinguishing AD from both the cohort of non-AD patients and specifically patients with CPUO. Beyond reduced numbers, CyTOF and RNA-seq analysis of both control and diseased NK cells demonstrated that AD-associated NK cells have a distinct transcriptional program indicative of AICD and a selective loss of a subset of mature CD56^{dim} NK cells. These findings suggest that chronic inflammation associated with AD can promote activation, maturation, and global loss of blood NK cells.

Studies over the past two decades have found various abnormalities in NK cell populations in the blood of patients with AD (13-16). However, the precise nature of these defects and their relationship to disease status has been unclear. These data are consistent with previous studies measuring decreased total CD56^{dim} NK cells in patients with AD (15), including one study that specifically observed a reduction in CD57⁺ NK cells (14). Furthermore, the increased apoptotic gene signature in CD56^{dim} NK cells is supported by a previous study showing that AD NK cells had enhanced apoptosis in vitro after phorbol. 12-myristate 13-acetate (PMA)/ionomycin stimulation (16). This preferential apoptotic response was dependent on the presence of donor monocytes (16), although the signals required for this interaction to induce apoptosis are not clear. It is possible that monocytes are a source of proinflammatory cytokines, such as IL-12 and IL-18, in the blood of patients with AD that may contribute to both the activated phenotype and enhanced sensitivity to AICD. However, the activating signals triggering NK cell death in vivo are unknown.

In addition to the total NK cell reduction and loss of mature CD56^{dim} cells, an outgrowth of a small, nonclassical population of CD56^{dim} cells that lacked canonical NK cell receptors was also observed, which was termed NCR⁻ cells. These cells are present at very low frequency in control subjects but expanded substantially in the setting of AD. The study design limited the ability to deeply phenotype or functionally characterize these cells because the multidimensional analysis used to identify them exceeds the number of parameters available for cell sorting. However, nonclassical populations of NK cells with reduced NCR expression and impaired lytic properties have been observed in humans with chronic viral infections such as human immunodeficiency virus and hepatitis C virus (52-54). These cells share several features with the NCR⁻ population observed in the dataset such as decreased NKp46, NKp80, and perforin expression. Thus, understanding the relationship between chronic inflammation, NK cell deficiency, and antiviral immunity in AD may provide insight into a common mechanism underlying their expansion in chronic diseases.

In conjunction with the loss of peripheral blood NK cells, an elevated NK cell transcriptional signature in lesional skin of patients with AD was identified. The presence and developmental origins of NK cells in healthy human skin remain poorly characterized. Current data support the presence of a population of CD3⁻ CD56⁺ CD16⁻ NK cells in healthy and inflamed skin that most closely resembles the CD56^{bright} population in the blood (55). Furthermore, a recent study identified a proliferative, skin-homing population of CD56^{bright} NK cells during acute dengue virus infection that represented most of the skin NK cells in these patients (56). However, whether a conventional population of CD56^{dim} CD16⁺ NK cells exists in human skin or traffics into the skin in response to noninfectious inflammatory conditions remains unclear. In mice, two populations of NK cells have been identified in the skin: tissue-resident and recirculating (57). An increased number and frequency of NK cells were identified in AD-like skin compared to control skin, which further increased after systemic IL-15 SA stimulation. Although the number of NK cells leaving the blood for the skin would likely be insufficient to account for the substantial loss in total NK cells in patients' blood, it was hypothesized that some degree of migration of NK cells from the blood to the skin supports the expanded numbers of NK cells in AD lesions and may represent an endogenous regulatory response to aberrant type 2 skin inflammation.

Here is evidence that the observed changes in peripheral blood NK cells, in addition to having diagnostic value, may have broader implications for both protective immunity and inflammation in the setting of chronic AD. In the preclinical model of AD, described here, it was found that a loss of NK cells in mice, through both genetic and pharmacologic approaches, resulted in an exacerbation of pathogenic ILC2 responses, suggesting that NK cells can regulate ILC2s in skin lesions. Although this study was limited to a single model system of AD, these findings are consistent with recent studies in the lung, showing that loss of NK cell-derived IFN-γ was accompanied by an increase in ILC2s and allergic lung inflammation (17). Therefore, these findings in the skin indicate that this NK cell-ILC2 inhibitory axis may be an evolutionarily conserved regulatory mechanism present at multiple-barrier surfaces. In light of these findings, the systemic loss of NK cells identified, here, in patients with AD may not only impair antiviral immunity in patients but also contribute to the unchecked type 2 inflammation and skin lesions. In addition, the restoration of NK cell numbers in patients after type 2 cytokine blockade with dupilumab indicates that NK cell reduction may occur secondary to allergic inflammation, creating a vicious disease cycle.

Although this study focused on the effects of inflammatory cytokine signaling on NK cell numbers, the ability of NK cells to limit AD-associated inflammation suggests that reduced NK cell numbers and/or function could be a predisposing risk factor for AD. In support of this perspective, a previous study of two separate European cohorts detected a potentially protective effect of KIR2DS1 against the development of AD (58). Another study found a single-nucleotide polymorphism (SNP) in KIR2DS2 associated with AD and asthma (59). Although these observations implicate NK cell homeostasis in AD and atopy (the genetic tendency to develop allergic diseases such as allergic rhinitis, asthma, and atopic dermatitis (eczema)), the functional relationship between these SNPs and NK cell function or disease outcomes is undefined. Furthermore, the current study is limited with respect to the severity of its study population. Although NK cell deficiency was found in moderate-to-severe patients, whether NK cells are reduced in the blood of patients with less severe symptoms or correlate with disease severity was not evaluated.

In a departure from the current immunosuppressive treatment approaches for AD, these findings offer a new paradigm in which reversing NK cell deficiency in patients may provide therapeutic benefit. NK cell agonism (49-51) and NK cell checkpoint inhibition (60) strategies in cancer immunotherapy have proven highly effective in both expanding host NK cells and boosting their function. Subcutaneous administration of the IL-15 SA complex ALT-803 has been well-tolerated in patients with cancer, with the main adverse events being an injection site reaction that resolves without intervention and transient hypertension (61). In another trial in which IL-15 SA complexes were given in combination with the checkpoint inhibitor nivolumab over the course of 6 months (49), IL-15 SA adverse events lessened over time, indicating that this may be a viable approach for chronic treatment. As such, NK cell-based immunotherapy can be used as a treatment approach for AD.

Materials and Methods

Study Design

The rationale for the design of human studies was to undertake either a case-control study approach or perform a basic observational study in a diseased population (cases) in response to a highly effective treatment (dupilumab) over time to determine whether blood NK cell populations are different in frequency, number, phenotype, and/or identity between cases and controls or in response to treatment. Given the observational nature of the translational studies, there was no randomization or formal blinding process for the investigators. Where possible, measurements were acquired in a blinded manner and then unblinded after results were obtained. Although there was no predefined power analysis performed, interim power analyses were performed when trends were observed, and additional cases and/or controls were added to achieve the determined cohort size. Sample acquisition was stopped upon reaching statistical significance (P<0.05).

The rationale for the design of murine studies was to use only enough mice in each experiment to observe a statistically significant difference between groups. These numbers were not based on predefined power analyses but previous experience with this well-validated model system and numbers of mice used previously (5, 45, 62). Age-, sex-, and strain-matched controls were always used. Whenever possible, measurements were acquired in a blinded manner and then unblinded after results were obtained. Interim power analyses were performed when trends were observed, and experiments were replicated to achieve the necessary cohort size. Data shown in the figures are representative of at least three independent experiments or pooled across experiments when a larger cohort size was required.

For retrospective analysis of blood lymphocyte populations, Lymphocyte-13 flow cytometry data was extracted from Cerner, a centralized data management software program used by the Barnes-Jewish Hospital (BJH) laboratory, for all patients seen at BJH between January 2015 and December 2018 [Institutional Review Board (IRB) no. 201703135]. Patients who were seen in an oncology clinic or who had a history of any malignancy were excluded. This resulted in a total of 363 patients designated as controls. The diagnosis of AD (cases) was made on the basis of the revised Hanifin and Rajka criteria (63). Patients with AD who visited the Washington University School of Medicine (WUSM) specialty itch clinic between January 2015 and December 2018, were ordered a Lymphocyte-13 laboratory test, and had an IGA score of ≥3 (moderate-to-severe diagnosis) were selected for inclusion in the analysis. This resulted in 25 moderate-to-severe AD cases. A total of 69 patients with CPUO (64) were represented in the control group and, in some analyses, were also compared directly against patients with AD. CPUO was diagnosed on the basis of the presence of chronic pruritus for >6 weeks in the absence of a primary skin rash, endocrine disease, metabolic disorders, uremia, hepatobiliary disease, malignancy, infection, neurologic disease, drug reactions, or psychiatric etiology (65, 66). This cohort was selected from patients who visited WUSM specialty itch clinic within the same study period and received a diagnosis of CPUO. These retrospective analyses of existing clinical data qualified for an informed consent waiver. Primary data are reported in data file S1.

Primary Human Sample Collection

Functional assays and RNA-seq on control NK cells were performed on peripheral blood obtained from Mohs surgery patients (IRB no. 201507042). After obtaining informed consent, blood and skin samples were obtained from patients with moderate-to-severe AD (IGA 3) seen in the Division of Dermatology at WUSM/BJH from November 2015 to September 2018 (IRB no. 201410014). CyTOF analysis was performed on PBMCs from healthy volunteers, following informed consent (IRB no. 201503172). All samples were obtained from peripheral blood draw, and PBMCs were isolated by Ficoll density gradient purification and frozen at −80° C. until assayed.

Cases and controls were age- and sex-matched whenever possible. However, two studies were not sex-matched (see e.g., FIG. 1, FIG. 2). These studies had opposite sex biases and found similar results, which were confirmed in separate, sex-matched analyses (see e.g., FIG. 12C), suggesting that these findings are not dependent on sex differences. Detailed characteristics of research cases and controls are in TABLES 1-7.

Research Animals

WT C56Bl/6J, Rag1^{-/-}, and Rosa-stop-floxed-DTA mice were initially purchased from The Jackson Laboratory and bred in house. Cd8^{-/-} mice were directly purchased from The Jackson Laboratory. Il15^{-/-} mice were originally generated by Kennedy and Peschon (46), obtained from Taconic, and bred in house. Ncr1-iCre mice were generated by E.V. (67) and bred in house. All experiments were conducted with the approval of the Washington University Institutional Animal Care and Use Committee. Animals were housed on a standard 12:12 light:dark cycle with free access to food and water. Experiments were performed on independent cohorts of male and female mice. For induction of AD-like disease, 8- to 12-week-old mice were treated with 1 nmol MC903 (Tocris Bioscience) in 10 μl of 100% ethanol (EtOH) vehicle, or vehicle alone, on the bilateral ear skin daily for 7 or 12 days. Bodyweight and ear thickness were measured daily with a digital scale and analog caliper by the same investigator. For tissue harvest, animals were euthanized by CO₂ inhalation.

Mass Cytometry

Mass cytometry was performed as previously described (48). Briefly, metal-tagged antibodies were purchased from Fluidigm or custom-conjugated using the Maxpar X8 Antibody Labeling Kit according to the manufacturer's instructions (Fluidigm). All antibodies were titrated before use. PBMCs were stained with metal-conjugated antibodies (TABLE 8) with the following protocol: PBMCs were washed and counted, and 3×10⁶ cells were stained with primary and then secondary surface antibodies for 30 min each on ice. Cells were then washed and stained with cisplatin for viability, fixed for 30 min on ice, permeabilized using the FoxP3 Transcription Factor Staining Kit (eBioscience) per the manufacturer's instructions, and left in CyTOF Cell Staining Buffer (Fluidigm) overnight. The next day, cells were repermeabilized, barcoded with Cell-ID 20-Plex Pd Barcoding Kit (Fluidigm), pooled, and stained with intracellular primary and secondary antibodies on ice for 30 min each. Last, Cell-ID Intercalator-Ir (Fluidigm) was added to detect nuclei. Cells were diluted in distilled deionized water containing 10% EQ Calibration Beads (Fluidigm) at 10⁶ cells per ml and acquired on a CyTOFII instrument (Fluidigm) at the Bursky Center for Human Immunology and Immunotherapy Programs Immunomonitoring Lab core facility. The data were randomized with Fluidigm acquisition software and normalized with MATLAB bead normalization (68).

TABLE 8
Human NK cell CyTOF antibodies.

| | | | | | | | S [symbol] g | |
|---|---|---|---|---|---|---|---|---|
| Antigen | Metal | Vendor | Cat No. | Clone | Dilution | Clust.? | Ar [symbol] h | Max |
| CD45 | [symbol] Y | Fluidigm | 30899 [symbol] B | HIB0 | 200 | Yes | 5 | 12000 |
| CD14 | 141 Pr | BO Pharmiagen | 555396 | M5H2 | 100 | | 5 | 12000 |
| KIR3O [symbol] | 143 Nd | R&D | | Z27 | 962 | Yes | 5 | 12000 |
| KIR2DS4 (CD158 [symbol] ) | 145 N [symbol] | Beckman Coulter | | FES172 | 100 | Yes | 5 | 12000 |
| KIP2DL) (CD158a) | 146 N [symbol] | BD Pharmiagen | | EB6B | 200 | Yes | 5 | 12000 |
| NKG2D | 147 N [symbol] | R&D | | 1D [symbol] | 50 | Yes | [symbol] | 1200 |
| KIR2DL2/2DL3 (CD158b) | 148 Nd | BD Pharmiagen | 559783 | CH-L | 56 [symbol] | Yes | 5 | 12000 |
| CD19 | 150 Nd | eBioscience | 14-0199-30 | HIB19 | 400 | | 5 | 12000 |
| TRAIL | 151 En | Biolegend | 308202 | RIK-2 | 200 | Yes | 2 | 1200 |
| CD8 | 152 [symbol] | Biolegend | 344727 | SL1 | 971 | Yes | 5 | 12000 |
| CD [symbol] 2L | 153 Eu | Fluidigm | 32530 [symbol] 4B | DREG- | 333 | Yes | 5 | 12000 |
| | | | | 58 | | | | |
| KIR2DL [symbol] (CD158 [symbol] ) | 154 Sm | Beckman Coulter | | UP-R1 | 200 | Yes | 5 | 12000 |
| CD27 | 155 Gd | Fluidigm | 31550 [symbol] B | [symbol] 28 | 333 | Yes | 5 | 12000 |
| CXCP4 | 156 Gd | Fluidigm | 315602 [symbol] B | 12 [symbol] | 100 | Yes | 5 | 12000 |
| CCR4 | 158 Gd | Fluidigm | 3156006A | 205410 | 333 | Yes | 5 | 12000 |
| NK92C | 159 Tb | R&D | MAB138 | 134591 | 400 | Yes | 5 | 12000 |
| CD69 | 160 Gd | Biolegend | 320939 | FN50 | [symbol] | Yes | 5 | 12000 |
| NKp [symbol] | 161 [symbol] | Biolegend | 325202 | P30-15 | 124 | Yes | 5 | 12000 |
| CD [symbol] | 163 [symbol] | Biolegend | 303502 | DX22 | 120 | Yes | 5 | 12000 |
| Tim-3 | 104 Dy | Biolegend | 3450 [symbol] 9 | F38-2E2 | 100 | Yes | 5 | 12000 |
| CD16 | 105 H [symbol] | Fluidigm | 316500 [symbol] B | JG8 | 333 | Yes | 5 | 12000 |
| NKG2A | 166 Er | Beckman Coulter | IM2750 | Z199 | 192 | Yes | 5 | 12000 |
| NKp44 | 167 Dy | Biolegend | 325101 | P44-8 | 204 | Yes | 2 | 12000 |
| CD226 ( [symbol] NAMI) | 168 Er | BD Pharmiagen | [symbol] 9787 | OX11 | 200 | Yes | 2 | 12000 |
| NKp80 | 170 Er | R&D | MAB1900 | 239127 | 149 | Yes | 5 | 12000 |
| CD57 | 172 Yb | Fluidigm | 3172009B | HCD57 | 200 | Yes | 5 | 12000 |
| CD3 | 173 Yb | eBioscience | 14-0038-82 | OC [symbol] T [symbol] | 1023 | | 5 | 12000 |
| NKp46 | 174 Yb | R&D | MAB1850 | 195314 | 400 | Yes | 2 | 12000 |
| CD56 (NCAM) | 17 [symbol] Yb | Fluidigm | 32700 [symbol] B | HCD36 | 333 | Yes | 5 | 12000 |
| CD12b | 209 Bi | Fluidigm | 3209 [symbol] B | JCRF44 | 500 | Yes | 2 | 1200 |
| FcERg-FITC | None | M [symbol] -Mark (EMD | FCAB8400F | | 50 | Yes | 5 | 12000 |
| | | Millipore) | | | | | | |
| T-Bet | 149 [symbol] | BO Pharmiagen | 361262 | 4B10 | 148 | Yes | 5 | 12000 |
| Ki67 | 162 Dy | Fluidigm | 3262012B | B56 | 1000 | Yes | 5 | 12000 |
| [symbol] omes | 169 Tm | Invitrogen | 14-4877-82 | WD1928 | 200 | Yes | 5 | 12000 |
| [symbol] mB | 171 Yb | Fluidigm | 3171002B | GB11 | 500 | Yes | 5 | 12000 |
| [symbol] | 175 L [symbol] | Fluidigm | 3175004B | BO4 [symbol] | 1000 | Yes | 5 | 12000 |
| Anti-F [symbol] TC | 144 Nd | Fluidigm | 3144006B | FTT-22 | 200 | | | |

[symbol]

indicates data missing or illegible when filed

Samples were debarcoded using the MATLAB Nolan laboratory single-cell debarcoder v0.2 (nolanlab/single-cell-debarcoder; GitHub) (69) as live, single cells (Bead⁻ Cisplatin⁻ DNA1/2⁺) and then imported into Cytobank. Samples were normalized to machine controls to reduce batch effects of multiple run days. To do this, pregated CD19⁻ CD14⁻ CD3⁻ CD56⁺ events were exported from Cytobank for each sample. Median signal intensities were extracted for each machine control, and a normalization vector was generated as the ratio of a machine control to a benchmark run using a custom code in R (doi:10.5281/zenodo.3568404). This normalization vector was then applied to each sample in the run, and the normalized files were reimported as a single experiment into Cytobank for further analysis. Dimensionality reduction was performed with Cytobank viSNE using equal sampling, 10,000 iterations, perplexity 50, and 6 of 0.5.

Flow Cytometry

For in vitro human studies, cells were harvested from cell culture, stained with primary antibodies on ice for 30 min, washed, stained with 7-AAD (BioLegend), and acquired on a BD Fortessa X-20. For animal studies, ear skin was digested in 500 μl of Liberase TL (0.25 mg/ml) (Roche) in Dulbecco's modified Eagle's medium (Sigma-Aldrich) at 37° C. and 5% CO₂ for 90 min. Skin and draining lymph nodes were then manually homogenized through a 70-μm cell strainer to obtain a single-cell suspension. All cells were stained with Zombie UV dye (BioLegend) for viability at room temperature for 20 min, followed by primary antibodies on ice for 30 min (TABLE 9). Secondary streptavidin-conjugated fluorophores were stained on ice for 30 min. Cells were then fixed with BD Cytoperm/Cytofix reagent on ice for 30 min or overnight at 4° C. before data acquisition on a BD LSRFortessa X-20 special order research product. Data were analyzed with FlowJo 10 (Tree Star).

TABLE 9
Flow cytometry antibodies.

| Species | Anitgen | Fluer | Vendor | Cat No. | Clone | Dilution |
|---|---|---|---|---|---|---|
| Human | CD45 | AF700 | Biolegend | 362514 | 2D [symbol] | 1/300 |
| Human | CD56 | PE/Cy7 | Biolegend | 318318 | HCD56 | 1/200 |
| Human | CD3 | FITC | Biolegend | 300306 | HIT [symbol] | 1/300 |
| Human | CD19 | FITC | Biolegend | 302206 | HIB19 | 1/300 |
| Human | Cleaved Caspase-3 | AF647 | BD Pharmiagen | 560626 | C92-605 | 5 [symbol] |
| Human | CD14 | FITC | Biolegend | 325604 | HCD14 | 1/300 |
| Human | CD34 | FITC | Biolegend | 343604 | 5 [symbol] | 1/399 |
| Human | CD16 | PE | Biolegend | 302056 | 3O8 | 1/399 |
| Human | CD56 | BV605 | Biolegend | 318333 | HCD56 | 1/200 |
| Mouse | CD45 2 | PE | Biolegend | 109808 | 104 | 1/300 |
| Mouse | NK1.1 | PE/Cy7 | Biolegend | 108714 | PK136 | 1/300 |
| Mouse | CD49b | APC | eBioscience | 17-5971-82 | DX5 | 1/200 |
| Mouse | CD11b | BV519 | Biolegend | 101245 | M [symbol] 70 | 1/300 |
| Mouse | CD127 | BV650 | Biolegend | 135043 | A [symbol] R34 | 1/100 |
| Mouse | ST2-biotin | None | Biolegend | 145307 | RMST2-2 | 1/300 |
| NA | Strepavidin | FITC | Biolegend | 405201 | | 1/1,000 |
| Mouse | CD3 [symbol] | PerCP/Cy5.5 | eBioscience | 45-0031-82 | 145-2C11 | 1/300 |
| Mouse | CD19 | PerCP/Cy5.5 | eBioscience | 45-0193-82 | 1D3 | 1/300 |
| Mouse | CD5 | PerCP/Cy5.5 | eBioscience | 45-0051-82 | 53-7.3 | 1/300 |
| Mouse | CD11 [symbol] | PerCP/Cy5.5 | eBioscience | 45-0114-82 | N418 | 1/300 |
| Mouse | F [symbol] RIα | PerCP/Cy5.5 | Biolegend | 134320 | MAR-1 | 1/399 |
| Mouse | KLRGI | PE/Dazzle | Biolegend | 138424 | 2F1/KLRG1 | 1/399 |
| Mouse | Siglec-F | BV421 | BD Horizon | 562681 | E [symbol] -2440 | 1/300 |

[symbol]

indicates data missing or illegible when filed

In Vitro Stimulation Assays

For the CD16 ligation assay, 0.5×10⁶ to 1×10⁶ PBMCs were stimulated in 96-well round-bottom plates in a 37° C. incubator with 5% CO₂. PBMCs were thawed and cultured for 12 to 14 hours with basal medium [RPMI 1640 containing 10% Human AB serum (Sigma-Aldrich), 10 mM Hepes (Corning), 1× nonessential amino acids (Corning), 1 mM sodium pyruvate (Corning), 1× penicillin (100 IU/ml)-streptomycin (100 μg/ml) solution (Gibco), 2 mM L-glutamine (Gibco)] and recombinant human IL-15 (rhIL-15) (1 ng/ml) (Miltenyi). Before stimulation, medium was changed to fresh medium containing indicated concentrations of anti-CD16 (BD Biosciences) antibody-conjugated MACS iBeads (Miltenyi). After 3 hours, cells were harvested for flow cytometric analysis as described above. For cytokine priming assays, replicates of 10⁶ PBMCs harvested from a healthy donor Leukopak (STEMCELL) were incubated overnight in basal medium containing rhIL-15 (1 ng/ml), rhIL-12 (10 ng/ml) (BioLegend), and rhIL-18 (100 ng/ml) (Gibco). For purified NK cell stimulation, NK cells were isolated from Leukopak PBMCs using the Human NK Cell Negative Selection Kit (STEMCELL) in a 96-well round-bottom plate using an EasyPlate magnet (STEMCELL) per the manufacturer's instructions.

IL-15 SA

IL-15 SA was administered by intraperitoneal injection of 1 μg of SA in 100 μl of phosphate-buffered saline (PBS) daily on days 4 to 7 of MC903 treatment. IL-15 SA was prepared as previously described (47). Briefly, 20 μg of recombinant murine IL-15 (rmIL-15; eBioscience or STEMCELL) was combined with 90 μg of a chimeric sIL-15Rα fused to the Fc domain of human IgG1 (R&D Systems) at a concentration of 0.1 mg/ml of IL-15 in PBS. The mixture was then vortexed, incubated at 37° C. for 20 min, and diluted to 10 μg/ml of IL-15 in PBS. SA concentration was calculated with reference to IL-15 for in vivo dosing. Stable loading was confirmed by measuring free rmIL-15 in solution after coincubation of rmIL-15 and IL-15Rα-Fc proteins by enzyme-linked immunosorbent assay (ELISA) (R&D Systems, DuoSet). Actual values based on a standard curve were comparable to values predicted based on molar ratios. Isotype control solution was prepared in the same fashion with rhIgG1 (R&D Systems), 0.1% bovine serum albumin, and 1 μM glycine in PBS. Aliquots of SA or isotype solution were frozen at −20° C. and thawed just before injection. Clinical scoring was adapted from the eczema area and severity index (EASI) (70), performed by a treatment-blinded investigator, and calculated as the sum of a redness score (0=none, 5=severe) and a scaling score (0=none, 5=severe).

Histological Analysis

For murine AD-like histopathology analysis, ear tissues were harvested on experimental day 12, fixed in 4% paraformaldehyde, and embedded in paraffin before sectioning and staining with hematoxylin and eosin (H&E). Slides were imaged using the NanoZoomer 2.0-HT System (Hamamatsu). Images were scored by a blinded investigator, and histopathology score was calculated as the sum of the following criteria: keratin thickness (average of 3 measurements per 40×image×3 images), epidermal thickness (average of 3 measurements per 40×image×3 images), epidermal spongiosis (0 to 3 rating), microabscesses (0 to 3 rating), vascular area (sum per treated side/1000), and inflammatory infiltrate (0 to 10 rating per whole ear).

Plasma Cytokine Measurements

Plasma was isolated from peripheral blood draw from AD or healthy control subjects by Ficoll gradient separation and frozen at −80° C. Plasma was diluted 1:1 in assay diluent and blocked by preincubation on Protein L-coated plates (Thermo Fisher Scientific) for 90 min at room temperature on an orbital shaker before the detection assay. Cytokines were measured using a 27-plex custom Luminex ELISA kit (R&D Systems), and data were collected on a FLEXMAP three-dimensional system (Thermo Fisher Scientific).

Quantitative Reverse Transcription Polymerase Chain Reaction of Murine Skin

MC903-treated, AD-like ear skin, and EtOH-treated control skin were harvested on day 12 of treatment, placed in RNAlater (Invitrogen) overnight at 4° C., and stored at −80° C. RNA was isolated after tissue homogenization with a bead homogenizer in buffer RLT (Qiagen) with 142 mM ß-mercaptoethanol using the RNeasy Mini Kit (Qiagen) per the manufacturer's instructions. Genomic DNA was removed with a TURBO DNA-Free kit (Invitrogen) before complementary DNA (cDNA) synthesis with the High Capacity cDNA Reverse Transcription Kit (Applied Biosystems). For relative quantification of Ifng mRNA, 10 ng of cDNA was used to perform quantitative polymerase chain reaction (qPCR) with a commercial primer-probe assay (assay ID Mm.PT.58.41769240; Integrated DNA Technologies) and the TaqMan Gene Expression Master Mix (Applied Biosystems) on a StepOnePlus machine (Applied Biosystems).

RNA-Seq of Skin

Murine MC903- and vehicle EtOH-treated skin RNA-seq data were obtained from a previously published study (45). For sequencing of human skin, 4-mm punch biopsies were placed in RNAlater (Life Technologies) overnight at 4° C. and stored at −80° C. until further processing. Skin was homogenized with a bead homogenizer in RNA lysis buffer, and RNA was isolated with the RNeasy Mini Kit (Qiagen). Library preparation, alignment, and transcript abundance were performed by the Genome Technology Access Center (GTAC) at WUSM as previously described (45). Briefly, after deoxyribonuclease treatment (TURBO DNase, Invitrogen), ribosomal RNA was removed with Ribo-Zero kit (MRZH11124; Illumina) and RNA was reverse-transcribed using SuperScript II RT enzyme (Invitrogen). Human samples were sequenced with an average of 60 million 1×50 single reads on an Illumina HiSeq3000. Reads were aligned to Ensembl release 76 human genome assembly using STAR (71), gene counts were determined with Subread:featureCount (72), and sequence performance was assessed with RSeQC (73).

RNA-Seq of Sort-Purified NK Cells

Live, CD45⁺ CD3⁻ CD56^{dim} NK cells (30,000 to 100,000) were sort-purified from cryopreserved PBMCs on an Aria II (BD Biosciences) into 200 μl of lysis buffer RA1 (Macherey-Nagel) containing tris(2-carboxyethyl)phosphine (TCEP) per the manufacturer's instructions. Cells were then vortexed for 30 s, frozen on dry ice, and stored at −80° C. RNA isolation was performed with the NucleoSpin RNA XS Kit (Macherey-Nagel). Library preparation, alignment, and transcript abundance were performed by the GTAC at WUSM. Ribosomal RNA was removed, and cDNA was generated with the SMARTer Kit (Clontech) with 10 ng of total RNA per sample. Samples were sequenced to an average depth of 34 million 1×50 reads on a HiSeq3000 (Illumina). Reads were aligned to Ensembl release 76 human genome assembly using STAR (71), gene counts were determined with Subread:featureCount (72), and sequence performance was assessed with RSeQC (73).

RNA-Seq Analyses

Genes were filtered for rowSums( )>10 counts and protein coding designation. Differential gene expression analysis was conducted using the Bioconductor package DESeq2 (74) in R v3.5.1 using default parameters. GSEAs were performed with GAGE (generally applicable gene set enrichment) (75) and GSEA (Broad Institute). Differentially enriched NK GO terms were grouped into larger biological categories based on keywords. Enrichment score for GO terms was calculated as the Stat.mean score divided by P value from GAGE output in R. Enrichment score for GO categories is the mean of the enrichment scores of GO terms within each category. Genes associated with GO terms were determined to be genes with differential expression between AD and control groups (P<0.05), with indicated differentially expressed GO assignments extracted using AnnotationDbi package (Bioconductor).

Statistical Analysis

Data are presented as means±SEM unless otherwise specified. Murine results are representative of at least two independent experiments. Graphical results and statistical testing for RNA-seq and retrospective laboratory testing analysis were conducted in R v3.5.1. Graphical results and statistical testing for remaining studies were conducted with GraphPad Prism 8. Data were tested for normality using the Shapiro-Wilk test, and a nonparametric test (Mann-Whitney U or Wilcoxon) was used for data that were deemed nonnormal. Otherwise, a t test or analysis of variance (ANOVA) was performed, where indicated. For tests with multiple comparisons, the Sidak (two-way ANOVA) or Sidak-Holm (t tests) correction for multiple comparisons was used. ROC curve analyses were conducted in SPSS v25.0 for Mac. ROC curve analyses were conducted assuming that a lower NK cell number was a positive test result.

### REFERENCES

1. R. J. Hay, N. E. Johns, H. C. Williams, I. W. Bolliger, R. P. Dellavalle, D. J. Margolis, R. Marks, L. Naldi, M. A. Weinstock, S. K. Wulf, C. Michaud, C. J. L. Murray, M. Naghavi, The global burden of skin disease in 2010: An analysis of the prevalence and impact of skin conditions. J. Invest. Dermatol. 134, 1527-1534 (2014). 2. C. Karimkhani, R. P. Dellavalle, L. E. Coffeng, C. Flohr, R. J. Hay, S. M. Langan, E. O. Nsoesie, A. J. Ferrari, H. E. Erskine, J. I. Silverberg, T. Vos, M. Naghavi, Global skin disease morbidity and mortality: An update from the global burden of disease study 2013. JAMA Dermatol. 153, 406-412 (2017). 3. A. M. Drucker, A. R. Wang, W.-Q. Li, E. Sevetson, J. K. Block, A. A. Qureshi, The burden of atopic dermatitis: Summary of a report for the national eczema association. J. Invest. Dermatol. 137, 26-30 (2017). 4. E. B. Brandt, U. Sivaprasad, Th2 cytokines and atopic dermatitis. J. Clin. Cell. Immunol. 2, 1-13 (2011). 5. B. S. Kim, M. C. Siracusa, S. A. Saenz, M. Noti, L. A. Monticelli, G. F. Sonnenberg, M. R. Hepworth, A. S. Van Voorhees, M. R. Comeau, D. Artis, TSLP elicits IL-33-independent innate lymphoid cell responses topromote skin inflammation. Sci. Transl. Med. 5, 170ra16 (2013). 6. M. Salimi, J. L. Barlow, S. P. Saunders, L. Xue, D. Gutowska-Owsiak, X. Wang, L.-C. Huang, D. Johnson, S. T. Scanlon, A. N. J. McKenzie, P. G. Fallon, G. S. Ogg, A role for IL-25 and IL-33-driven type-2 innate lymphoid cells in atopic dermatitis. J. Exp. Med. 210, 2939-2950 (2013). 7. B. Roediger, R. Kyle, K. H. Yip, N. Sumaria, T. V. Guy, B. S. Kim, A. J. Mitchell, S. S. Tay, R. Jain, E. Forbes-Blom, X. Chen, P. L. Tong, H. A. Bolton, D. Artis, W. E. Paul, B. F. de St Groth, M. A. Grimbaldeston, G. Le Gros, W. Weninger, Cutaneous immunosurveillance and regulation of inflammation by group 2 innate lymphoid cells. Nat. Immunol. 14, 564-573 (2013). 8. D. Simon, L. R. Braathen, H.-U. Simon, Eosinophils and atopic dermatitis. Allergy 59, 561-570 (2004). 9. F.-T. Liu, H. Goodarzi, H.-Y. Chen, IgE, mast cells, and eosinophils in atopic dermatitis. Clin. Rev. Allergy Immunol. 41, 298-310 (2011). 10. J. Spergel, A. S. Paller, Atopic dermatitis and the atopic march. J. Allergy Clin. Immunol. 112, S118-S127 (2003). 11. S. M. Langan, K. Abuabara, S. E. Henrickson, O. Hoffstad, D. J. Margolis, Increased risk of cutaneous and systemic infections in atopic dermatitis—A cohort study. J. Invest. Dermatol. 137, 1375-1377 (2017). 12. R. J. M. Engler, J. Kenner, D. Y. M. Leung, Smallpox vaccination: Risk considerations for patients with atopic dermatitis. J. Allergy Clin. Immunol. 110, 357-365 (2002). 13. B. Mourad, N. Abdelnabi, L. H. Elgarhy, M. Attia, Peripheral natural killer cell subsets in atopic dermatitis. J. Egypt. Women's Dermatol. Soc. 12, 129-135 (2015). 14. W. Wehrmann, U. Reinhold, S. Kukel, N. Franke, M. Uerlich, H. W. Kreysel, Selective alterations in natural killer cell subsets in patients with atopic dermatitis. Int. Arch. Allergy Appl. Immunol. 92, 318-322 (1990). 15. C. Luci, C. Gaudy-Marqueste, P. Rouzaire, S. Audonnet, C. Cognet, A. Hennino, J.-F. Nicolas, J.-J. Grob, E. Tomasello, Peripheral natural killer cells exhibit qualitative and quantitative changes in patients with psoriasis and atopic dermatitis. Br. J. Dermatol. 166, 789-796 (2012). 16. M. Katsuta, Y. Takigawa, M. Kimishima, M. Inaoka, R. Takahashi, T. Shiohara, NK cells and γδ+ T cells are phenotypically and functionally defective due to preferential apoptosis in patients with atopic dermatitis. J. Immunol. 176, 7736-7744 (2006). 17. J. Bi, L. Cui, G. Yu, X. Yang, Y. Chen, X. Wan, NK cells alleviate lung inflammation by negatively regulating group 2 innate lymphoid cells. J. Immunol. 198, 3336-3344 (2017). 18. A. B. Molofsky, F. Van Gool, H.-E. Liang, S. J. VanDyken, J. C. Nussbaum, J. Lee, J. A. Bluestone, R. M. Locksley, Interleukin-33 and interferon-γ counter-regulate group 2 innate lymphoid cell activation during immune perturbation. Immunity 43, 161-174 (2015). 19. K. Moro, H. Kabata, M. Tanabe, S. Koga, N. Takeno, M. Mochizuki, K. Fukunaga, K. Asano, T. Betsuyaku, S. Koyasu, Interferon and IL-27 antagonize the function of group 2 innate lymphoid cells and type 2 innate immune responses. Nat. Immunol. 17, 76-86 (2016). 20. J. S. Silver, J. Kearley, A. M. Copenhaver, C. Sanden, M. Mori, L. Yu, G. H. Pritchard, A. A. Berlin, C. A. Hunter, R. Bowler, J. S. Erjefalt, R. Kolbeck, A. A. Humbles, Inflammatory triggers associated with exacerbations of COPD orchestrate plasticity of group 2 innate lymphoid cells in the lungs. Nat. Immunol. 17, 626-635 (2016). 21. B. S. Kim, T. G. Berger, G. Yosipovitch, Chronic Pruritus of Unknown Origin (CPUO): Uniform Nomenclature and Diagnosis as a Pathway to Standardized Understanding and Treatment. J. Am. Acad. Dermatol. 81, 1223-1224 (2019). 22. M. A. Cooper, T. A. Fehniger, M. A. Caligiuri, The biology of human natural killer-cell subsets. Trends Immunol. 22, 633-640 (2001). 23. A. J. Wilk, C. A. Blish, Diversification of human NK cells: Lessons from deep profiling. J. Leukoc. Biol. 103, 629-641 (2018). 24. D. M. Strauss-Albee, J. Fukuyama, E. C. Liang, Y. Yao, J. A. Jarrell, A. L. Drake, J. Kinuthia, R. R. Montgomery, G. John-Stewart, S. Holmes, C. A. Blish, Human NK cell repertoire diversity reflects immune experience and correlates with viral susceptibility. Sci. Transl. Med. 7, 297ra115 (2015). 25. J. A. Wagner, M. Rosario, R. Romee, M. M. Berrien-Elliott, S. E. Schneider, J. W. Leong, R. P. Sullivan, B. A. Jewell, M. Becker-Hapak, T. Schappe, S. Abdel-Latif, A. R. Ireland, D. Jaishankar, J. A. King, R. Vij, D. Clement, J. Goodridge, K.-J. Malmberg, H. C. Wong, T. A. Fehniger, CD56bright NK cells exhibit potent antitumor responses following IL-15 priming. J. Clin. Invest. 127, 4042-4058 (2017). 26. P. L. Collins, M. Cella, S. I. Porter, S. Li, G. L. Gurewitz, H. S. Hong, R. P. Johnson, E. M. Oltz, M. Colonna, Gene regulatory programs conferring phenotypic identities to human NK cells. Cell 176, 348-360.e12 (2018). 27. N. K. Bjökström, P. Riese, F. Heuts, S. Andersson, C. Fauriat, M. A. Ivarsson, A. T. Björklund, M. Flodström-Tullberg, J. Michaëlsson, M. E. Rottenberg, C. A. Guzmán, H.-G. Ljunggren, K.-J. Malmberg, Expression patterns of NKG2A, KIR, and CD57 define a process of CD56dim NK-cell differentiation uncoupled from NK-cell education. Blood 116, 3853-3864 (2010). 28. C. M. Nielsen, M. J. White, M. R. Goodier, E. M. Riley, Functional significance of cd57 expression on human nk cells and relevance to disease. Front. Immunol. 4, 422 (2013). 29. A. Muntasell, A. Pupuleku, E. Cisneros, A. Vera, M. Moraru, C. Vilches, M. López-Botet, Relationship of NKG2C copy number with the distribution of distinct cytomegalovirus induced adaptive NK cell subsets. J. Immunol. 196, 3818-3827 (2016). 30. A. Muntasell, C. Vilches, A. Angulo, M. López-Botet, Adaptive reconfiguration of the human NK-cell compartment in response to cytomegalovirus: A different perspective of the host-pathogen interaction. Eur. J. Immunol. 43, 1133-1141 (2013). 31. H. Schlums, F. Cichocki, B. Tesi, J. Theorell, V. Beziat, T. D. Holmes, H. Han, S. C. C. Chiang, B. Foley, K. Mattsson, S. Larsson, M. Schaffer, K.-J. Malmberg, H.-G. Ljunggren, J. S. Miller, Y. T. Bryceson, Cytomegalovirus infection drives adaptive epigenetic diversification of NK cells with altered signaling and effector function. Immunity 42, 443-456 (2015). 32. I. Hwang, T. Zhang, J. M. Scott, A. R. Kim, T. Lee, T. Kakarla, A. Kim, J. B. Sunwoo, S. Kim, Identification of human NK cells that are deficient for signaling adaptor FcRγ and specialized for antibody-dependent immune functions. Int. Immunol. 24, 793-802 (2012). 33. H. Spits, D. Artis, M. Colonna, A. Diefenbach, J. P. Di Santo, G. Eberl, S. Koyasu, R. M. Locksley, A. N. J. Mckenzie, R. E. Mebius, F. Powrie, E. Vivier, Innate lymphoid cells—A proposal for uniform nomenclature. Nat. Rev. Immunol. 13, 145-149 (2013). 34. L. Chen, Y. Youssef, C. Robinson, G. F. Ernst, M. Y. Carson, K. A. Young, S. D. Scoville, X. Zhang, R. Harris, P. Sekhri, A. G. Mansour, W. K. Chan, A. P. Nalin, H. C. Mao, T. Hughes, E. M. Mace, Y. Pan, N. Rustagi, S. S. Chatterjee, P. H. Gunaratne, G. K. Behbehani, B. L. Mundy-Bosse, M. A. Caligiuri, A. G. Freud, CD56 expression marks human group 2 innate lymphoid cell divergence from a shared NK cell and group 3 innate lymphoid cell developmental pathway. Immunity 49, 464-476.e4 (2018). 35. L. Krabbendam, M. Nagasawa, H. Spits, S. M. Bal, Isolation of human innate lymphoid cells. Curr. Protoc. Immunol. 122, e55 (2018). 36. L. A. Beck, D. Thaçi, J. D. Hamilton, N. M. Graham, T. Bieber, R. Rocklin, J. E. Ming, H. Ren, R. Kao, E. Simpson, M. Ardeleanu, S. P. Weinstein, G. Pirozzi, E. Guttman-Yassky, M. Suárez-Fariñas, M. D. Hager, N. Stahl, G. D. Yancopoulos, A. R. Radin, Dupilumab treatment in adults with moderate-to-severe atopic dermatitis. N. Engl. J. Med. 371, 130-139 (2014). 37. D. Thaçi, E. L. Simpson, L. A. Beck, T. Bieber, A. Blauvelt, K. Papp, W. Soong, M. Worm, J. C. Szepietowski, H. Sofen, M. Kawashima, R. Wu, S. P. Weinstein, N. M. H. Graham, G. Pirozzi, A. Teper, E. R. Sutherland, V. Mastey, N. Stahl, G. D. Yancopoulos, M. Ardeleanu, Efficacy and safety of dupilumab in adults with moderate-to-severe atopic dermatitis inadequately controlled by topical treatments: A randomised, placebo-controlled, dose-ranging phase 2b trial. Lancet 387, 40-52 (2016). 38. E. L. Simpson, T. Bieber, E. Guttman-Yassky, L. A. Beck, A. Blauvelt, M. J. Cork, J. I. Silverberg, M. Deleuran, Y. Kataoka, J.-P. Lacour, K. Kingo, M. Worm, Y. Poulin, A. Wollenberg, Y. Soo, N. M. H. Graham, G. Pirozzi, B. Akinlade, H. Staudinger, V. Mastey, L. Eckert, A. Gadkari, N. Stahl, G. D. Yancopoulos, M. Ardeleanu; SOLO 1 and SOLO 2 Investigators, Two phase 3 trials of dupilumab versus placebo in atopic dermatitis. N. Engl. J. Med. 375, 2335-2348 (2016). 39. J. R. Ortaldo, A. T. Mason, J. J. O'Shea, Receptor-induced death in human natural killer cells: Involvement of CD16. J. Exp. Med. 181, 339-344 (1995). 40. M. A. Smith, M. Maurin, H. I. Cho, B. Becknell, A. G. Freud, J. Yu, S. Wei, J. Djeu, E. Celis, M. A. Caligiuri, K. L. Wright, PRDM1/Blimp-1 controls effector cytokine production in human NK cells. J. Immunol. 185, 6058-6067 (2010). 41. R. Romee, B. Foley, T. Lenvik, Y. Wang, B. Zhang, D. Ankarlo, X. Luo, S. Cooley, M. Verneris, B. Walcheck, J. Miller, NK cell CD16 surface expression and function is regulated by a disintegrin and metalloprotease-17 (ADAM17). Blood 121, 3599-3608 (2013). 42. A. M. Newman, C. L. Liu, M. R. Green, A. J. Gentles, W. Feng, Y. Xu, C. D. Hoang, M. Diehn, A. A. Alizadeh, Robust enumeration of cell subsets from tissue expression profiles. Nat. Methods 12, 453-457 (2015). 43. M. Li, P. Hener, Z. Zhang, S. Kato, D. Metzger, P. Chambon, Topical vitamin D3 and low-calcemic analogs induce thymic stromal lymphopoietin in mouse keratinocytes and trigger an atopic dermatitis. Proc. Natl. Acad. Sci. U.S.A. 103, 11736-11741 (2006). 44. M. C. Siracusa, B. S. Kim, J. M. Spergel, D. Artis, Basophils and allergic inflammation. J. Allergy Clin. Immunol. 132, 789-801 (2013). 45. L. K. Oetjen, M. R. Mack, J. Feng, T. M. Whelan, H. Niu, C. J. Guo, S. Chen, A. M. Trier, A. Z. Xu, S. V. Tripathi, J. Luo, X. Gao, L. Yang, S. L. Hamilton, P. L. Wang, J. R. Brestoff, M. L. Council, R. Brasington, A. Schaffer, F. Brombacher, C.-S. Hsieh, R. W. Gereau IV, M. J. Miller, Z.-F. Chen, H. Hu, S. Davidson, Q. Liu, B. S. Kim, Sensory neurons co-opt classical immune signaling pathways to mediate chronic itch. Cell 171, 217-228.e13 (2017). 46. M. K. Kennedy, M. Glaccum, S. N. Brown, E. A. Butz, J. L. Viney, M. Embers, N. Matsuki, K. Charrier, L. Sedger, C. R. Willis, K. Brasel, P. J. Morrissey, K. Stocking, J. C. Schuh, S. Joyce, J. J. Peschon, Reversible defects in natural killer and memory CD8 T cell lineages in interleukin 15-deficient mice. J. Exp. Med. 191, 771-780 (2000). 47. Y. Guo, L. Luan, W. Rabacal, J. K. Bohannon, B. A. Fensterheim, A. Hernandez, E. R. Sherwood, IL-15 superagonist-mediated immunotoxicity: Role of NK cells and IFN-γ. J. Immunol. 195, 2353-2364 (2015). 48. R. Romee, M. Rosario, M. M. Berrien-Elliott, J. A. Wagner, B. A. Jewell, T. Schappe, J. W. Leong, S. Abdel-Latif, S. E. Schneider, S. Willey, C. C. Neal, L. Yu, S. T. Oh, Y.-S. Lee, A. Mulder, F. Claas, M. A. Cooper, T. A. Fehniger, Cytokine-induced memory-like natural killer cells exhibit enhanced responses against myeloid leukemia. Sci. Transl. Med. 8, 357ra123 (2016). 49. J. M. Wrangle, V. Velcheti, M. R. Patel, E. Garrett-Mayer, E. G. Hill, J. G. Ravenel, J. S. Miller, M. Farhad, K. Anderton, K. Lindsey, M. Taffaro-Neskey, C. Sherman, S. Suriano, M. Swiderska-Syn, A. Sion, J. Harris, A. R. Edwards, J. A. Rytlewski, C. M. Sanders, E. C. Yusko, M. D. Robinson, C. Krieg, W. L. Redmond, J. O. Egan, P. R. Rhode, E. K. Jeng, A. D. Rock, H. C. Wong, M. P. Rubinstein, ALT-803, an IL-15 superagonist, in combination with nivolumab in patients with metastatic non-small cell lung cancer: A nonrandomised, open-label, phase 1b trial. Lancet Oncol. 19, 694-704 (2018). 50. M. A. Geller, L. A. Bendzick, C. Ryan, S. Chu, A. Lenvik, A. P. N. Skubitz, K. L. M. Boylan, R. Isaksson Vogel, J. Miller, M. Felices, Combination therapy with IL-15 superagonist (ALT-803) and PD-1 blockade enhances human NK cell immunotherapy against ovarian cancer. Gynecol. Oncol. 145, 19 (2017). 51. J. S. Miller, S. Cooley, S. Holtan, M. Arora, C. Ustun, E. Jeng, H. C. Wong, M. R. Verneris, J. E. Wagner, D. J. Weisdorf, B. R. Blazar, T. A. Fehniger, R. Romee, ‘First-in-human’ phase I dose escalation trial of IL-15N72D/IL-15Rα-Fc superagonist complex (ALT-803) demonstrates immune activation with anti-tumor activity in patients with relapsed hematological malignancy. Blood 126, 1957 (2015). 52. D. Mavilio, G. Lombardo, J. Benjamin, D. Kim, D. Follman, E. Marcenaro, M. A. O'Shea, A. Kinter, C. Kovacs, A. Moretta, A. S. Fauci, Characterization of CD56−/CD16+ natural killer (NK) cells: A highly dysfunctional NK subset expanded in HIV-infected viremic individuals. Proc. Natl. Acad. Sci. U.S.A. 102, 2886-2891 (2005). 53. N. K. Björkström, H.-G. Ljunggren, J. K. Sandberg, CD56 negative NK cells: Origin, function, and role in chronic viral disease. Trends Immunol. 31, 401-406 (2010). 54. V. D. Gonzalez, K. Falconer, J. Michaëlsson, M. Moll, O. Reichard, A. Alaeus, J. K. Sandberg, Expansion of CD56− NK cells in chronic HCV/HIV-1 co-infection: Reversion by antiviral treatment with pegylated IFNα and ribavirin. Clin. Immunol. 128, 46-56 (2008). 55. F.-D. Shi, H.-G. Ljunggren, A. La Cava, L. Van Kaer, Organ-specific features of natural killer cells. Nat. Rev. Immunol. 11, 658-671 (2011). 56. C. L. Zimmer, M. Cornillet, C. Solà-Riera, K.-W. Cheung, M. A. Ivarsson, M. Q. Lim, N. Marquardt, Y.-S. Leo, D. C. Lye, J. Klingström, P. A. MacAry, H.-G. Ljunggren, L. Rivino, N. K. Björkström, NK cells are activated and primed for skin-homing during acute dengue virus infection in humans. Nat. Commun. 10, 3897 (2019). 57. D. K. Sojka, B. Plougastel-Douglas, L. Yang, M. A. Pak-Wittel, M. N. Artyomov, Y. Ivanova, C. Zhong, J. M. Chase, P. B. Rothman, J. Yu, J. K. Riley, J. Zhu, Z. Tian, W. M. Yokoyama, Tissue-resident natural killer (NK) cells are cell lineages distinct from thymic and conventional splenic NK cells. eLife 3, e01659 (2014). 58. W. Niepiekło-Miniewska, E. Majorczyk, Ł. Matusiak, K. Gendzekhadze, I. Nowak, J. Narbutt, A. Lesiak, P. Kuna, J. Ponińska, A. Pietkiewicz-Sworowska, B. Samoliński, R. Płoski, J. C. Szepietowski, D. Senitzer, P. Kuśnierczyk, Protective effect of the KIR2DS1 gene in atopic dermatitis. Gene 527, 594-600 (2013). 59. D. Vukcevic, J. A. Traherne, S. Ness, E. Ellinghaus, Y. Kamatani, A. Dilthey, M. Lathrop, T. H. Karlsen, A. Franke, M. Moffatt, W. Cookson, J. Trowsdale, G. McVean, S. Sawcer, S. Leslie, Imputation of KIR types from SNP variation data. Am. J. Hum. Genet. 97, 593-607 (2015). 60. P. André, C. Denis, C. Soulas, C. Bourbon-Caillet, J. Lopez, T. Arnoux, M. Bléry, C. Bonnafous, L. Gauthier, A. Morel, B. Rossi, R. Remark, V. Breso, E. Bonnet, G. Habif, S. Guia, A. I. Lalanne, C. Hoffmann, O. Lantz, J. Fayette, A. Boyer-Chammard, R. Zerbib, P. Dodion, H. Ghadially, M. Jure-Kunkel, Y. Morel, R. Herbst, E. Narni-Mancinelli, R. B. Cohen, E. Vivier, Anti-NKG2A mAb is a checkpoint inhibitor that promotes anti-tumor immunity by unleashing both T and NK cells. Cell 175, 1731-1743.e13 (2018). 61. R. Romee, S. Cooley, M. M. Berrien-Elliott, P. Westervelt, M. R. Verneris, J. E. Wagner, D. J. Weisdorf, B. R. Blazar, C. Ustun, T. E. DeFor, S. Vivek, L. Peck, J. F. DiPersio, A. F. Cashen, R. Kyllo, A. Musiek, A. Schaffer, M. J. Anadkat, I. Rosman, D. Miller, J. O. Egan, E. K. Jeng, A. Rock, H. C. Wong, T. A. Fehniger, J. S. Miller, First-in-human phase 1 clinical study of the IL-15 superagonist complex ALT-803 to treat relapse after transplantation. Blood 131, 2515-2527 (2018). 62. B. S. Kim, K. Wang, M. C. Siracusa, S. A. Saenz, J. R. Brestoff, L. A. Monticelli, M. Noti, E. D. Tait Wojno, T. C. Fung, M. Kubo, D. Artis, Basophils promote innate lymphoid cell responses in inflamed skin. J. Immunol. 193, 3717-3725 (2014). 63. R. Sidbury, W. L. Tom, J. N. Bergman, K. D. Cooper, R. A. Silverman, T. G. Berger, S. L. Chamlin, D. E. Cohen, K. M. Cordoro, D. M. Davis, S. R. Feldman, J. M. Hanifin, A. Krol, D. J. Margolis, A. S. Paller, K. Schwarzenberger, E. L. Simpson, H. C. Williams, C. A. Elmets, J. Block, C. G. Harrod, W. Smith Begolka, L. F. Eichenfield, Guidelines of care for the management of atopic dermatitis: Section 4. Prevention of disease flares and use of adjunctive therapies and approaches. J. Am. Acad. Dermatol. 71, 1218-1233 (2014). 64. N. K. Mollanazar, M. Sethi, R. V. Rodriguez, L. A. Nattkemper, F. V. Ramsey, H. Zhao, G. Yosipovitch, Retrospective analysis of data from an itch center: Integrating validated tools in the electronic health record. J. Am. Acad. Dermatol. 75, 842-844 (2016). 65. A. Z. Xu, S. V. Tripathi, A. L. Kau, A. Schaffer, B. S. Kim, Immune dysregulation underlies a subset of patients with chronic idiopathic pruritus. J. Am. Acad. Dermatol. 74, 1017-1020 (2016). 66. G. W. M. Millington, A. Collins, C. R. Lovell, T. A. Leslie, A. S. W. Yong, J. D. Morgan, T. Ajithkumar, M. J. Andrews, S. M. Rushbook, R. R. Coelho, S. J. Catten, K. Y. C. Lee, A. M. Skellett, A. G. Affleck, L. S. Exton, M. F. Mohd Mustapa, N. J. Levell, British Association of Dermatologists' guidelines for the investigation and management of generalized pruritus in adults without an underlying dermatosis, 2018. Br. J. Dermatol. 178, 34-60 (2018). 67. E. Narni-Mancinelli, J. Chaix, A. Fenis, Y. M. Kerdiles, N. Yessaad, A. Reynders, C. Gregoire, H. Luche, S. Ugolini, E. Tomasello, T. Walzer, E. Vivier, Fate mapping analysis of lymphoid cells expressing the NKp46 cell surface receptor. Proc. Natl. Acad. Sci. U.S.A. 108, 18324-18329 (2011). 68. R. Finck, E. F. Simonds, A. Jager, S. Krishnaswamy, K. Sachs, W. Fantl, D. Pe'er, G. P. Nolan, S. C. Bendall, Normalization of mass cytometry data with bead standards. Cytometry A 83A, 483-494 (2013). 69. E. R. Zunder, R. Finck, G. K. Behbehani, E. D. Amir, S. Krishnaswamy, V. D. Gonzalez, C. G. Lorang, Z. Bjornson, M. H. Spitzer, B. Bodenmiller, W. J. Fantl, D. Pe'er, G. P. Nolan, Palladium-based mass tag cell barcoding with a doublet-filtering scheme and single-cell deconvolution algorithm. Nat. Protoc. 10, 316-333 (2015). 70. J. M. Hanifin, M. Thurston, M. Omoto, R. Cherill, S. J. Tofte, M. Graeber, EASI Evaluator Group, The eczema area and severity index (EASI): Assessment of reliability in atopic dermatitis. Exp. Dermatol. 10, 11-18 (2001). 71. A. Dobin, C. A. Davis, F. Schlesinger, J. Drenkow, C. Zaleski, S. Jha, P. Batut, M. Chaisson, T. R. Gingeras, STAR: Ultrafast universal RNA-seq aligner. Bioinformatics 29, 15-21 (2013). 72. Y. Liao, G. K. Smyth, W. Shi, featureCounts: An efficient general purpose program for assigning sequence reads to genomic features. Bioinformatics 30, 923-930 (2014). 73. L. Wang, S. Wang, W. Li, RSeQC: Quality control of RNA-seq experiments. Bioinformatics 28, 2184-2185 (2012). 74. M. I. Love, W. Huber, S. Anders, Moderated estimation of fold change and dispersion for RNA-seq data with DESeq2. Genome Biol. 15, 550 (2014). 75. W. Luo, M. S. Friedman, K. Shedden, K. D. Hankenson, P. J. Woolf, GAGE: Generally applicable gene set enrichment for pathway analysis. BMC Bioinformatics 10, 161 (2009).

What is claimed is:

1. A method of increasing an NK cell population or function in a subject having an allergic disorder, comprising administering an NK cell-stimulating agent to the subject in an amount effective to
(i) increase an NK cell level or function in the subject compared to the NK cell level or function in a control not having the allergic disorder;
(ii) increase the NK cell level or function in the subject compared to the NK cell level or function of the subject before being administered the NK cell-stimulating agent; or
(iii) increase the NK cell level to a level greater than 97.5 percentile.

2. The method of claim 1, wherein increasing NK cell level or function in the subject treats or prevents symptoms associated with the allergic disorder.

3. The method of claim 1, wherein the allergic disorder is associated with NK cell level or function depletion.

4. The method of claim 1, wherein the allergic disorder is selected from atopic dermatitis (AD), eczema, food allergy, asthma, an eosinophilic esophagitis or eosinophilic gastrointestinal disorder, a deficiency in type 1 immunity, allergic rhinitis, chronic rhinosinusitis, or a related allergic disorder thereof.

5. The method of claim 1, wherein the allergic disorder is atopic dermatitis (AD).

6. The method of claim 1, wherein the subject has less than a 97.5 percentile level of NK cells before being administered the NK cell-stimulating agent.

7. The method of claim 1, wherein the NK cell-stimulating agent comprises an IL-15 agonist, an IL-15 superagonist, or a combination thereof.

8. The method of claim 1, wherein the NK cell-stimulating agent is an IL-15 superagonist.

9. The method of claim 1, wherein the NK cell-stimulating agent is not dupilumab or IL-15.

10. The method of claim 1, wherein the NK cell-stimulating agent increases the NK cell level or function in the subject to a level above 97.5 percentile.

11. The method of claim 1, wherein the NK cell-stimulating agent is administered in an amount effective to prevent or ameliorate symptoms of the allergic disorder.

12. The method of claim 11, wherein ameliorating symptoms of the allergic disorder comprises:
reducing redness and scaling (clinical score 0-5);
reducing Numerical Rating scale (NRS) itch score;
reducing Investigator Global Assessment (IGA) score; or
reducing inflammatory, AD-associated serum biomarkers, TARC (CCL17), IL-4, or IL-13.

13. The method of claim 5, wherein the NK cell-stimulating agent is administered in an amount effective to ameliorate symptoms associated with atopic dermatitis (AD).

14. The method of claim 13, wherein ameliorating symptoms of atopic dermatitis (AD) comprises reducing erythema (redness), scaling, blood eosinophilia, serum IgE, or itch behavior (pruritus).

15. The method of claim 1, wherein the NK cell-stimulating agent is administered in an amount effective to improve histopathologic features selected from one or more of the group consisting of acanthosis (epidermal thickening), hyperkeratosis (stratum corneum thickening), spongiosis (epidermal edema), and mixed dermal lymphocyte and eosinophil infiltration.

16. The method of claim 1, wherein the NK cell-stimulating agent induces NK cell expansion in a dose-dependent manner.

17. The method of claim 1, wherein the NK cell-stimulating agent comprises an NK cell checkpoint inhibitor.

18. The method of claim 1, wherein the NK cell-stimulating agent comprises an IL-32 inhibiting agent, an IL-32α inhibiting agent, an IL-4 inhibiting agent, an IL-4 receptor α inhibiting agent, an IL-13 inhibiting agent, or an IL-13 receptor α inhibiting agent, or a combination thereof.

19. The method of claim 1, wherein the NK cell-stimulating agent comprises:
an IL-15 agonist, an IL-15 superagonist, or a combination thereof; and
an IL-32α inhibiting agent, an IL-32 inhibiting agent, an IL-4 inhibiting agent, an IL-4 receptor α inhibiting agent, an IL-13 inhibiting agent, or an IL-13 receptor α inhibiting agent, or a combination thereof.

20. The method of claim 8, wherein the IL-15 superagonist is selected from an IL-15:sIL-15Rα complex; a receptor-linker-IL-15 (RLI), a fusion polypeptide of IL-15 and IL-15Rα Sushi domain; ALT-803, a complex of IL-15 mutant IL-15N72D and a Sushi domain of IL-15Rα; or a combination thereof.

21. The method of claim 19, wherein
the IL-32 inhibiting agent is an anti-IL-32 mAb;
the IL-32α inhibiting agent an anti-IL-32α mAb;
the IL-4 inhibiting agent is an anti-IL-4 mAb;
the IL-4 receptor α inhibiting agent is an anti-IL-4 receptor α mAb;
the IL-13 inhibiting agent is an anti-L-13 mAb; or
the IL-13 receptor α inhibiting agent is an anti-IL-13 mAb.

22. The method of claim 1, wherein the NK cell-stimulating agent is a bispecific monoclonal antibody capable of simultaneously enhancing IL-15 activity and reducing IL-32α activity, IL-32 activity, IL-4 activity, IL-4 receptor α activity, IL-13 activity, or IL-13 receptor α activity.

23. The method of claim 1, wherein the NK cell-stimulating agent is a monoclonal antibody or bispecific monoclonal antibody comprising one or more of the group consisting of: an IL-15 agonist, an IL-15 superagonist, an IL-32α inhibiting agent, an IL-32 inhibiting agent, an IL-4 inhibiting agent, an IL-4 receptor α inhibiting agent, an IL-13 inhibiting agent, or an IL-13 receptor α inhibiting agent, or a combination thereof.

24. The method of claim 1, wherein increasing the NK cell population comprises increasing total NK cell population.

25. The method of claim 1, wherein increasing the NK cell population comprises increasing mature CD56^{dim} NK cell levels.

26. The method of claim 1, wherein a therapeutically effective amount of an NK cell-stimulating agent increases NK cell function before administration of the NK cell-stimulating agent.

27. The method of claim 1, wherein NK cell levels or NK cell function is measured in a sample comprising blood, optionally, peripheral blood.

28. The method of claim 1, further comprising administering a type 2 cytokine blockade therapy, optionally, dupilumab, to the subject.
